(19) United States
(12) Patent Application Publication
(10) Pub. No.: US 2005/0176029 A1
(43) Pub. Date: Aug. 11, 2005

(54) Nanoscale transduction systems for detecting molecular interactions

(75) Inventors: Michael Heller, San Diego, CA (US); Benjamin Sullivan, La Jolla, CA (US); Sanja Zlatanovic, La Jolla, CA (US); Sadik Esener, Solana Beach, CA (US); Dietrich Dehlinger, San Diego, CA (US)

Correspondence Address: BURNS, DOANE, SWECKER & MATHIS, LLP, 402 WEST BROADWAY, SUITE 400, SAN DIEGO, CA, 92101, US

(73) Assignee: The Regents of the University of California, Oakland, CA (US)

(21) Appl. No.: 10/970,756

(22) Filed: Oct. 20, 2004

Related U.S. Application Data
(60) Provisional application No. 60/513,042, filed on Oct. 20, 2003.

(51) Int. Cl. C12Q 1/68; G01N 33/53; H01L 21/00; G01N 33/543

(52) U.S. Cl. 435/6; 435/7.1; 436/523; 438/1

(57) ABSTRACT

The present invention relates to nanoscale transduction systems that produce reversible signals to facilitate detection. In one respect, the invention relates to the analysis of molecular binding events using higher order signaling nanoscale constructs, or “nanomachines”, that allow nanostructures to be individually detectable, even in the midst of high background noise. Such systems are particularly useful for improving the performance of rare target detection methods, as well as being generally useful in any field in which sensitivity, discrimination and confidence in detection are important.

### TECHNICAL FIELD

The present invention relates to nanoscale transduction systems that produce reversible signals to facilitate detection. In one respect, the invention relates to the analysis of molecular binding events using higher order signaling nanoscale constructs, or “nanomachines”, that allow nanostructures to be individually detectable, even in the midst of high background noise.

### BACKGROUND OF THE INVENTION

The photonics field includes a variety of emerging and converging technologies relating to light emission transmission, reflection, amplification and detection. The ability to harness and generate light and other forms of radiant energy has produced an equally broad variety of instrumentation, such as optical components and instruments, lasers and other light sources, fiber optics, electro-optical devices, and related hardware and electronics. Even with such an array of technologies and instrumentation, advances in photonics are still limited by the ways in which signal input is efficiently translated into meaningful output.

Considerable progress has been made in the development of signaling nanostructures, such as quantum dots, photonic crystal structures and metallic nanoparticles, which have improved fluorescent, luminescent or other optical properties. This progress has led to the development of nanostructures that can be easily detected with a conventional fluorescent microscope and charge-coupled device (CCD) imaging systems. These advances have enabled the signaling information obtained from populations of nanostructures in a given spatial field to flow from the nano/microscale to the macroscale level, which has made them useful in a variety of different applications.

In photonic systems designed to detect relatively low levels of signal, the true “signal” generated from the structure or event being monitored must be distinguishable from the background “noise” that is inherent in any photonic system. The inability to make such a distinction limits the use of conventional photonic systems to differentiate between the occurrence of a rare event and random noise. The use of “individually” detectable signaling nanostructures still does not solve the problem, because in many applications, the translation of signal from the nanostructures to a useable form is still not reliable. In particular, it has been difficult to capitalize on the intrinsic detectability of nanostructures to reliably differentiate the occurrence of specific molecular interactions such as single target molecule binding events and those due to nonspecific binding.

A considerable number of different techniques and assay formats are currently under development which utilize the unique signaling properties of nanostructures in a variety of different chemical and biological applications. Such applications utilize more complex “second generation” nanostructures with more sophisticated binding and signaling elements. For example, U.S. Pat. No. 6,630,307 discloses the use of semiconductor nanocrystals (quantum dots) as detectable labels that emit distinct wavelengths of light. In addition, U.S. Pat. No. 6,777,186 discloses the use of metallic nanoparticles and other reporter groups to create signaling probes or mechanisms. Despite such efforts, the underlying fundamental reasons for the loss of intrinsic sensitivity seem not to have been overcome, and single molecule detectability is not retained under most assay conditions.

Accordingly, there is a need for the development of higher order nanoscale transduction systems that are capable of demonstrating single molecule detectability. Such systems are provided by the present invention in the form of the “nanomachines” described herein and utilize a reversible signaling mechanism which is ideally suited, e.g., for the detection of rare molecular binding events.

### SUMMARY OF THE INVENTION

The present invention relates to higher order nanoscale transduction systems (i.e. “nanomachines”) that are capable of demonstrating single molecule detectability. One aspect of the present invention is a method that involves binding a nanostructure and an associated structure to a target and reversibly altering interaction between the nanostructure, the associated structure and the target. The reversible alteration may be in response to applied energy, such as: an electric field, a DC field, an AC field, capacitive field, thermal energy, electrical energy, chemical energy (e.g., adenosine triphosphate (ATP) or nicotinamide adenine dinucleotide (NADH)), etc. The energy may additionally be photonic, magnetic, kinetic, acoustic, ultrasonic, microwave or radiative.

The reversible alteration may take many different forms, such as deformation (elastic, inelastic, or plastic), as well as angular motion, a separation distance, a rotation, a linear displacement, helical motion, and the like. The alteration may additionally be in response to shear force or pressure.

The altered interaction between the components of the nanomachine may take the form of resonant energy (dipole coupling or quadrupole coupling), which may also be fluorescence resonance energy transfer (FRET). Alternatively, the interaction may be plasmonic, near field coupling, photonic, capacitive, magnetic or electrostatic.

The method may additionally include the step of detecting a spatially independent, temporally varying characteristic resulting from the interaction, which may be a variation in surface enhanced Raman scattering (SERS) or Raman spectra, luminescence, fluorescence, optical properties, color, magnetic field, electric field, temperature, etc. In one embodiment, the reversibly altering interaction between the nanostructure, the associated structure and the target is spatially independent. Additionally, the interaction may take place in solution or on a solid surface (microarrary or nanoarray). The interaction may take place with or without a priori knowledge of the spatial location. For example, the method may be either a homogeneous or heterogeneous assay.

In another embodiment, the present invention relates to an apparatus with a nanostructure and an associated structure, wherein the nanostructure and the associated structure are adapted to reversibly interact with each other and a target. Suitable nanostructures and associated structures include, for example, quantum dots, semiconductor nanoparticles, photonic crystals, metallic nanoparticles, ceramic nanoparticles, polymeric nanoparticles and nanotubes. In addition, the associated structure may include a signaling element, such as a fluorophore, a quencher, a chromophore, a phycobillic protein, a lumiphore, a fluorescent protein.

The apparatus may additionally include an interaction amplifying element, which may be attached to either the nanostructure or the associated structure. Such amplifying element may, for example, be a pressure responsive element or a displacement amplifying element.

The nanostructure of the apparatus may have attached thereto a fluorescent donor, in which case the associated structure may include a fluorescent quencher. Alternatively, the nanostructure and the associated structure may have attached thereto individual members of a fluorescent energy transfer (FRET) pair.

In another embodiment, the nanostructure and the associated structure are adapted to reversibly and spatially independently interact with each other and a target, which may occur in solution, or on a surface, such as on a microarray or nanoarray.

In yet another embodiment, the invention is a method including the steps of providing nanostructures, associated structures, and targets; and detecting a temporally varying, spatially independent, information signal produced by the nanostructures, associated structures, and targets. This method may additionally include the step of applying energy to the nanostructures, associated structures, and targets, which may be photonic, electrical, thermal, magnetic, periodic, a series of impulses, a single impulse, or be constant. The information signal may be a variation in fluorescence, color, temperature, electric field strength or magnetic field strength, or it may be a change in frequency of a characteristic of the nanostructure, associated structure, and target combinations. The method may further involve processing the detected information signal to classify a molecular binding event, for example utilizing neural networks, Bayesian networks or M-ary detection. The method may also include applying a driving force that produces a reversibly altering interaction between a nanostructure, an associated structure, and a target comprising the information signal.

A further embodiment of the present invention is a system for detecting a target that is adapted to produce a reversibly altering interaction between the nanostructure, associated structure and target; and also includes an input source adapted to impart energy or driving force to the nanostructure, associated structure, and target combination thereby producing the reversibly altering interaction; and a detector configured to detect transduced output generated by the reversibly altering interaction. Other aspects of the system, such as the imparted energy and the transduced output are as described above.

In addition to the aforementioned embodiments, the present invention includes a signaling nanostructure with at least one target binding region and at least one signal influencing region, wherein the signal influencing region has attached thereto a signal influencing element that alters a signaling characteristic of the nanostructure, and wherein the target binding region is selective for a predetermined target. The signal influencing element may be a signal inhibiting element. Additionally, the signaling nanostructure may be fluorescent and the signal inhibiting element may be a fluorescent quencher.

In one variation of this nanostructure, the target binding region and the signal influencing region are asymmetrically patterned on the surface of the signaling nanostructure. The nanostructure may contain one or many target binding regions. Additionally, the signal influencing element may be a metallic nanoparticle.

In another variation, the signaling nanostructure may have at least one signal influencing region having attached thereto a signal influencing element that alters a signaling characteristic of the nanostructure, wherein the signal influencing element may also have at least one target binding region having attached thereto a target binding element, and wherein the target binding element is selective for a predetermined target.

The target binding element in any of these embodiments or variations may be any member of a binding pair, such as an oligonucleotide, an antibody or a polypeptide. The signaling nanostructure and the signal influencing element may also be attached via the target binding element, which in this embodiment serves the function of acting as a bridge between structures.

In yet another variation, the signaling nanostructure has a first signal influencing element attached thereto, wherein the signal influencing element also includes at least one target binding region, and wherein the signaling nanostructure also includes a second signal influencing element attached thereto.

The present invention also includes kits that contain: a first signaling nanostructure having a first metallic nanoparticle attached thereto, wherein the first metallic nanoparticle also includes at least one first target binding region having attached thereto a first target binding element, and wherein the first target binding element is selective for a predetermined target; and a second metallic nanoparticle with at least one second target binding region having attached thereto a second target binding element, and wherein the second target binding element is selective for the same predetermined target. In such kits, the target binding elements may be as described above.

A variation on the kit is when the first and second metallic nanoparticles are attached via a tethering group, which may be a synthetic polymer, a single stranded nucleic acid, a fatty acid, a glycosaminoglycan or a polypeptide.

A still further embodiment of the present invention is a method with the steps of: binding a nanostructure to a target; binding an associated structure to the target; reversibly altering an interaction between the nanostructure, the associated structure and the target to produce information; and detecting the information. The target may be, for example, a nucleic acid, a protein, an inorganic surface, genomic nucleic acid, etc. It may also be from a biological sample, such as a cell or tissue sample.

In this exemplary method, the nanostructure may also include a first target binding region having a target binding element attached thereto that is selective for a predetermined target; and wherein the associated structure also includes a second target binding region having attached thereto a second target binding element that is selective for the same predetermined target. In one variation, both the first and second binding element may be nucleic acids or antibodies.

One embodiment of this method is a homogeneous assay, in which case, the method is adapted to be performed in a solution, and the detection step is performed without removing the nanostructure or the associated structure from the solution. Another embodiment of this method is an in situ assay, in which case, the method is adapted to be performed within a cellular structure, and the detection step is performed without removing the nanostructure or the associated structure from the cellular structure.

The present invention also includes a metallic nanoparaticle having attached thereto a signaling element and a target binding element, where the signaling element may be, for example, a quantum dot, a fluorophore, a FRET donor or a FRET acceptor.

In still another embodiment, the present invention includes a method for identifying a target nucleic acid molecule in a sample including the steps of: contacting the target nucleic acid molecule with a first nucleic acid probe having a signaling element attached thereto, wherein the first nucleic acid probe hybridizes to the target molecule; contacting the target nucleic acid with a second nucleic acid probe having a signal inhibiting element attached thereto, wherein the second probe hybridizes to the target nucleic acid molecule such that the signal inhibiting element is in proximity to the signaling element thereby reducing the signal associated with the signaling element; applying a pulsed electric field to a nucleic acid complex formed by the target nucleic acid and hybridized probes, wherein the pulsed electric field periodically interrupts the ability of the signal inhibiting element to reduce the signal associated with the signaling element thereby producing an oscillating signal; and detecting the oscillating signal. Other variations of this method are discussed below in Example 2, which include the ability to use the method to assemble a diagnostic profile that is correlated with a wild-type state, a pathological condition or a genetic alteration.

Other aspects of the invention are discussed throughout the specification.

### BRIEF DESCRIPTION OF THE DRAWINGS

FIG. 1 depicts a block diagram of a nanoscale transduction system (i.e. a nanomachine) of the present invention.

FIG. 2 depicts a basic embodiment of the nanomachine.

FIG. 3 depicts the nanomachine of FIG. 2 demonstrating light being emitted as a spherical wave in all directions.

FIG. 4 depicts a nanomachine utilizing a nanostructure with signal influencing elements attached to a signal influencing region on the surface of the nanoparticle.

FIG. 5 depicts a nanomachine employing signal influencing elements that function as a nanolens.

FIG. 6 depicts a nanomachine employing an interaction amplifying element (open square) attached to a Quencher/FRET probe.

FIG. 7 depicts a nanomachine employing a displacement amplifying element, which is the loop structure on the Quencher/FRET probe that serves as a “hinge”.

FIG. 8 depicts a nanomachine with a nanostrcutre and an associated structure, each having a signal influencing element, such as a metallic nanoparticle (grey circle) attached thereto.

FIG. 9 depicts a mechanistic analog of a linear nanomechanical element.

FIG. 10 depicts a simulation showing the electric field confinement and enhancement between to 50 nm gold nanoparticles, such as the plasmonic beads shown in FIG. 9.

FIGS. 11 to 17 depict further alternative embodiments of the nanomachines of the present invention as described in the “Exemplary Embodiments.”

FIG. 18 depicts the information flow from a field of nanostructures.

FIG. 19 depicts the three orders of magnitude increase in electric field concentration that is computationally observed within the cleft of the metallic nanoparticles.

FIG. 20 depicts the information flow from a field of nanomachines within a fluorescent in situ hybridization assay (FISH).

FIG. 21 depicts the addition of a complementary, one base mismatch, and two base mismatch associated structure (quencher probe) to nanostructures (quantum dots) indicating the ability to place the quantum dots in the “off” state.

FIG. 22 depicts the electric field effect of a representative embodiment of the present invention.

FIG. 23 depicts the ability to differentiate between complements, one base pair mismatches and two base pair mismatches.

FIGS. 24 to 27 are described below in Example 2.

### DESCRIPTION OF THE INVENTION

The present invention relates to nanoscale transduction systems that produce reversible signals to facilitate detection. In one respect, the invention relates to the analysis of molecular binding events using third generation signaling nanoscale constructs, or “nanomachines”, that allow nanostructures to be individually detectable, even in the midst of high background noise. Such systems are particularly useful for improving the performance of rare target detection methods, as well as being generally useful in any field in which sensitivity, discrimination and confidence in detection are important.

Conventional Approaches to Optimizing Molecular Assays

Traditional molecular assays rely upon time and concentration to traverse the free energy landscape of molecular binding assays. However, when searching for inherently rare molecules, sufficient progression towards thermodynamic equilibrium may require several hours of incubation. To complicate matters, efforts to drive assay kinetics with increases in probe concentration also favor the occupation of local minima, which create an inverse relationship between sensitivity and specificity, and an inverse relationship between speed and specificity. Furthermore, randomness within complex biological samples may alter the free energy landscape to stabilize improperly bound probes, preventing uniform washing, thermal, or salt stringency measures from discriminating between probes which have located a target, and those probes which are contained within similar but nonspecific energy minima.

These factors set fundamental limits of detection for assays designed to detect targets that are present in very low numbers, which includes assays based on antibodies, fluorescent probes, molecular beacons, PCR amplification, and so on. These factors also explain why assays that perform admirably under ideal laboratory conditions break down when presented with complex biological samples.

Accordingly, the majority of recent work in the assay development has focused on improving the sensitivity of detection. This includes enzyme amplification strategies, beaded fiber optic assemblies, direct and indirect electrochemical detection, a wealth of optical techniques such as SERS, quartz microbalances, nanoparticle accumulation assays, and a plurality of lesser known techniques such as exploiting DNA tethered ion channels. A particularly compelling development in the field of molecular detection was the advent of “individually detectable” fluorescent nanoparticles. Even though unresolvable below the diffraction limit of light, nanoparticles such as quantum dots enabled relatively inexpensive microscopy systems to detect signatures from objects only a few nanometers across.

However, the increased sensitivity of semiconductor based biomarkers was insufficient to overcome the low signal-to-noise ratio encountered in assays such as clinical genotyping assays. While many tests showed confident detection under ideal laboratory conditions, the performance of these same assays decreased considerably when applied to complex biological samples. In an effort to ameliorate the complexity, front-end sample preparation systems (i.e., separation, purification and amplification of the target molecules) were coupled to the detection methods. This resulted in the current atmosphere of labor intensive, time consuming and expensive procedures that are subject to unacceptable rates of false positives and false negatives.

Complicating matters are the formidable performance criteria for clinical genotyping applications. In order to be widely accepted, platforms must demonstrate speed, sensitivity and absolute specificity. This is especially true for infectious disease, forensics, cancer and bioterror agent detection, where false readings can have particularly unwelcome results. Molecular diagnostics are also important in drug discovery target validation, i.e. toxicity and cell signaling studies, where sensitivity and speed are of the utmost importance. If possible, automation of detection would also serve a great purpose in reducing the cost of new drug development, for stratifying the patient population as entrance criteria in clinical trials, and reducing the side effects for the general public by increasing the efficacy of prescribed pharmaceuticals.

First and Second Generation Conventional Nanoparticle Assays

Generally, most photonic nanoparticles have intrinsic properties that allow them to be individually detected with a conventional fluorescent microscope/CCD imaging system (i.e., “intrinsic detectability”). For example, nanoparticles in the form of fluorescent polymer (latex and polystyrene) nanobeads (20-500 nm) are commercially available, and can be individually detected with a conventional fluorescent microscope. In comparison, small (˜1 nm or less) organic chromophore/fluorophore molecules (fluorescein, Rhodamine, etc.) are not “individually detectable” or resolvable with such systems. Even though they can be “individually detected” with special near-field optical systems, such systems are not practical in most conventional laboratory settings.

Although in the technical sense, nanoparticles can be “individually detected” (observed in a field with minimal background noise), they cannot actually be “resolved” by the microscope system because their size is below the diffraction limit of light. This property is somewhat analogous to how stars can be individually detected with a telescope as point sources of light, even though they are not actually resolved. Unfortunately, whether using the more conventional nanoparticles, or the newer generation of photonic nanoparticles, it has still been difficult to capitalize on their intrinsic detectability to reliably signal the occurrence of specific molecular interactions, such as a single molecule binding event. Nevertheless, many researchers continue to make attempts to develop nanoparticle probe-based assays with improved specificity and sensitivity using various sensor technologies, both in homogeneous assays (performed in solution) and heterogeneous assays (bound to a solid phase). See, e.g., U.S. Pat. Nos. 6,777,186; 6,773,884; 6,767,702; 6,750, 016; 6,682,895; and 6,778,316).

By way of example, Fritzsche et al. describe a heterogeneous assay using gold nanoparticle probes (“probes”) that are attached to spatially defined locations (“test sites”) on a microarray surface (Fritzsche W., Taton, T. A., Nanotechnology, 14(12): R63-R73 (2003)). In this example, detailed atomic force microscope images of nanoparticle arrays were taken, and clearly demonstrated binding not just between matched DNA targets in the test sites, but also between misplaced probes, residual proteins, and surfaces between test sites.

As with any array-based system (microarray or nanoarray), the information is contained within a nonrandom, spatially contained area. A test-site at a given (x,y) coordinate on the array must be observed for an increase in nanoparticle density in order to detect and identify the target. The signal from that area must be compared to an area of the same size that does not have target molecules, but has been exposed to the nanoparticle probes (i.e, the negative control). This has at least two disadvantages: first, it increases the effective diffusion length for rare probes to reach the target, which dramatically slows the assay to require a day or more to complete; and second, relative comparisons establish fundamental signal to noise limits, and it is usually well above the level for single molecule detection, or even for low copy number detection (<1000 copies of target).

Most existing nanoparticle or microarray techniques rely upon thermodynamic equilibrium to ensure that specific probes will bind completely to their proper target sequence. For rare targets, this is insufficient to overcome the problems associated with complex DNA or protein samples, where many probes have similar binding free energies, as hybridization efficiency is strongly concentration dependent (Bhanot G., Louzoun Y., Zhu J., DeLisi C. Arrays. Biophysical Journal, 84: 124-135 (2003)). Therefore, many researchers also rely upon washing or thermal stringency to provide increased specificity (e.g., U.S. Pat. Nos. 6,773,884; 6,048,690; and 5,849,486).

While further washing may remove more nonspecific background, it also removes specific targets because of the overlap in the free energy between matched and mismatched probes. Thus the cost of improved specificity is a loss of sensitivity. In the case of single base discrimination analysis for point mutations or single nucleotide polymorphisms (genotyping), infectious disease phenotyping with antibodies, or target validation studies, very high stringency conditions are absolutely necessary for specificity. (The term “stringency” refers to the conditions under which hybridization takes place. The stringency of hybridization is determined by a number of factors during hybridization and during the washing procedure, including temperature, ionic strength, base composition, probe length, and concentration of formamide. These factors are outlined in, for example, Maniatis et al., 1982 and Sambrook et al., 1989.) Since these tests are of considerable importance for human diagnostics, specificity cannot be sacrificed for sensitivity because any false positives or false negative are not acceptable for most of these applications. Attempts to circumscribe the limitations to passive detection methods have met with limited success.

U.S. Pat. Nos. 6,602,400; 4,787,963 and 5,605,662 describe the use of an electric field to achieve a concentration effect to drive hybridization reactions. This has provided some leverage for the hybridization kinetic problem in microarray formats by using test-sites that have an underlying microelectrode (U.S. Pat. No. 6,048,690). The underlying microelectrode provides an electric field that causes the target molecules (negatively charged DNA molecules) to migrate and concentrate at the specific test-site. The field can also be reversed, providing a type of electric field stringency for removal of nonspecifically bound probes from the surface of the microarray. While providing some improvement in sensitivity and selectivity, low copy number detection is still not achieved with these microelectronic arrays, and as a result, these techniques required that DNA targets be preamplified via PCR prior to analysis.

U.S. Pat. Nos. 6,048,690 and 6,403,317 describes the perturbation of a fluorescent signal in a classical microarray system. Because the hybridization product is necessarily attached to the electronic stringency control device, the kinetics of hybridization are compromised and noise limits are imposed. Accordingly, the signal being monitored is being emitted from a population of upwards of several million fluorescently labeled probe molecules in order to overcome these limits, which required PCR amplification of the DNA prior to analysis.

Careful inspection of most detection methodologies reveals that the necessity to preamplify DNA targets is generally the case for heterogeneous assays and homogeneous formats alike. This roadblock seems to occur regardless of whether or not the probes are designed with nanoparticles or with conventional fluorophores, electrochemical, chemiluminescent, or radioisotope labels. Despite the ideal sensitivities of various detection methods, amplification of DNA target is often required even in cases where moderate numbers of target sequences (1,000 to 100,000 copies) are present, but the samples are complex (high amounts of extraneous DNA, proteins and other materials).

The problem of retaining intrinsic detectability of nanoparticle probes also seems not to be resolved when simple fluorescent resonant energy transfer (FRET) mechanisms are created between donor and acceptor (or quencher) probes. Because the quenching or energy coupling of FRET mechanisms is not perfect, these probes fail to eliminate background noise, despite their increased specificity.

Overall, the aforementioned factors set fundamental limits of detection for assays based on nanoparticles, fluorescent probes, molecular beacons, and so on. These factors also explain why assays which perform admirably under ideal laboratory conditions break down when presented with complex biological samples. Thus, the underlying reasons for the loss of intrinsic nanoparticle sensitivity seem not to have been overcome, and single molecule detectability is not retained under most assay conditions.

General Description of the Invention

The present invention relates to a “paradigm shifting” approach to enhancing the detection of molecular binding events, which results in improved performance (confidence, sensitivity, etc.) of molecular binding assays. This approach relies on the detection and processing of reversible signaling and the analysis of frequency characteristics of the signal, rather than the amplitude differences described in the aforementioned examples.

More particularly, the current invention relates to a nanoscale transduction system that circumvents the inverse relationships between speed, sensitivity and specificity that are characteristic of passive molecular assays. By attaching a nanostructure and an associated structure to the target thereby forming a “nanomachine”, and reversibly altering the interaction between the nanostructure, the associated structure and the target, this invention maximizes the information flow from the nanoscale to the macroscale to enable confident detection. As each nanomachine is endowed with a mechanistic ability to produce a time varying signal indicative of its immediate environment, the application of temporal signal processing theories can be used to extract specifically bound nanostructures amidst a field of nonspecifically bound nanostructures. As both the production and detection of the molecular signal are correlated random variables, multiple observations of the produced signal improve the confidence and sensitivity of detection over time. Furthermore, different frequency components of the signal carry information about whether the nanostructures are specifically, or nonspecifically bound. Traditional assays do not make use of this dynamic, and therefore are subject to the fundamental limits of background noise described above. Secondly, adding temporal information flow to nanostructures allows independence of detection on the whereabouts of the probe of interest. Probes no longer need to be attached to a specific test-site in a microarray, which causes dramatically increased kinetics in heterogeneous assays, and more strikingly, the ability to perform solution phase assays, or even combined homogeneous/heterogeneous assays where a high concentration of probes drive capture kinetics in solution, and then are brought to a surface for analysis.

The detection of periodic signals among high backgrounds has been widely studied in disciplines such as radar, communications theory, and astronomy. The extension of this stochastic detection theory to other fields, such as the life sciences, has been limited by the static nature of molecular binding assays (e.g., molecular diagnostics). By coupling a dynamic component to the characterization of nanostructures when used, e.g., as molecular probes, information about the immediate environment of the molecular probe could flow from the nano/microscale to the macroscale, despite high levels of corruptive background noise.

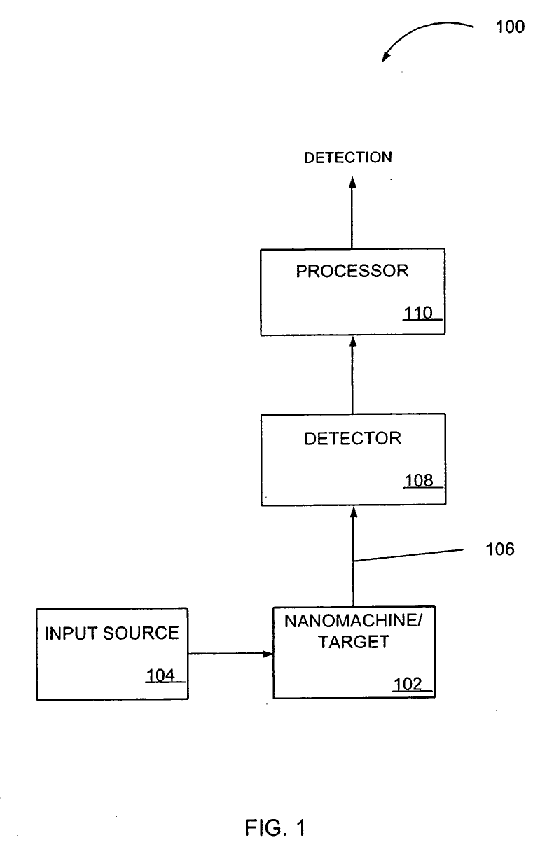

FIG. 1 is a block diagram of a nanoscale transduction system 100 that detects molecular interactions, as described above. In the system 100, a nanomachine/target combination 102 receives a driving source of energy from an input source 104. The nanomachine/target combination includes a nanostructure and an associated structure, each attached to a target, as described above.

The energy from the input source 104 can include, for example, electrical energy, thermal energy, optical energy, magnetic energy, or other physical phenomenon sufficient to impart energy to the nanomachine such that interaction between the nanostructure, the associated structure, and the target is reversibly altered in response to the energy. The energy from the input source 104 that is imparted to the nanomachine/target combination can be periodic, such as characterized by a sine wave, or it can be an impulse or a series of impulses of energy, or it can be a constant source of energy.

As described above, the reversible alteration can be one of many different types of alteration. For example, the reversible alternation can include a varying linear or angular distance, a rotation, a plastic deformation, an elastic deformation, a torsion, or a mutual attraction, such as magnetic, ionic or electrical attraction.

The reversible alteration in the nanomachine produces a change in a characteristic of the nanomachine that comprises a transduced output 106. The transduced output is detected by a detector 108. The transduced output can comprise, for example, a change, or variation, in nanomachine fluorescence, luminescence, color, dipole orientation, temperature, electrical field strength and frequency, or magnetic field strength.

The detector 108 can detect the changed characteristic (transduced output 106). The detector can operate to detect, for example, optical changes, including luminescence or color, of the nanomachine, thermal changes, electrical field, or magnetic field changes. The detector can include, for example, a camera such as CCD or CMOS type detector arrays, a photomultiplier tube (PMT), avalanche photodiode (APD), electrical or magnetic energy detection devices, or thermal detection devices.

The processor 110 receives output from the detector 108 and can process the detector output to identify changed characteristics of the nanomachine/target 102 that comprise phenomena for which detection is desired. The processing of the detector output can be performed with a variety of signal processing techniques, which will be known to those skilled in the art. For example, “MatLab” is a software application available from The Mathworks, Inc. of Natick, Mass., USA that is suitable for performing the data processing to identify meaningful detector output that indicates the presence of a desired target. For example, the processor 110 may include classification, neural networks, Baysian networks, or maximum a priori probability (MAP) detection.

The “Higher Order” Assays of the Invention

The signaling nanostructures of the present invention form higher order “nanomachines”, which are able to modulate or gate the signal from a basic photonic nanostructure to which a specific target molecule is bound. This is accomplished by designing a mechanistic property into the system which allows, upon binding of a target molecule, for a secondary structure to become associated as to proximate an element that influences the basic photonic nanostructure. The “nanomachine” is further designed such that input of another energy source (DC field, etc.) causes the distance to increase between the influencing group of the associated structure and the basic photonic nanostructure. Thus, the secondary energy input can be used to specifically modulate or gate those signaling nanostructures to which a target molecule has bound.

A basic nanostructure, such as a quantum dot, is “intrinsically detectable”, because it can be detected using conventional microscopic detection systems. Second generation nanostuctures are more complex and incorporate different binding members and signaling elements, while still being “intrinsically detectable”, they usually do not enable most assays with single molecule sensitivity.

Suitable nanostructures for use in the present invention include, e.g., quantum dots, semiconductor nanoparticles, photonic crystal nanostructures, metallic nanoparticles, ceramic nanoparticles, polymeric nanoparticles, nanotube structures (carbon nanotubes, etc.), fluorescent or luminescent macromolecules (dendrites, etc.), biological macromolecules including fluorescent proteins (e.g. phycobiliproteins) or luminescent proteins (e.g. luciferase), and photosynthetic macromolecular antenna structures. Such nanostructures are described in the literature and can be constructed using known methods.

Other suitable nanostructures include: nanoshells as disclosed in U.S. Pat. No. 6,344,272, metal colloids as disclosed in U.S. Pat. No. 5,620,584 272, fullerenes and derivatized fullerenes, as disclosed in U.S. Pat. Nos. 5,739,376; 6,162,926; 5,994,410, as well as nanotubes including single walled nanotubes, as disclosed in U.S. Pat. No. 6,183,714, all of which can also be derivatized.

In one embodiment, the nanostructures further comprise a target binding region, which in one embodiment involves derivatization to render the nanostructure competent to bind to target molecules and other elements. Such derivatization may include the attachment of a target binding element. However, it should be understood that one of the advantages of the present invention is that, unlike most molecular probes that require “specific” binding to the exact target molecule of interest to facilitate detection, it is assumed in this invention that even under high stringency conditions that there will be some number of mismatched DNA or nonspecifically bound proteins to the nanostructures. Accordingly, the present invention circumvents the necessity for absolute or even relative target-specificity, because by using the methods described herein, target bound nanostructures can be resolved from nonspecifically bound nanostructures.

Exemplary optional derivatizations to attach target binding elements include, inter alia, the attachment of DNA, RNA, polynucleotides, oligonucleotides, peptide nucleic acids (pNAs), or other DNA analogues, antibodies, proteins, peptides, or any other specific bio/chem/metal ligand or binding member that is capable of binding to the target molecule.

The “nanomachine” of the present invention also includes an associated structure that influences the photonic energy transfer from the nanostructure, such that the energy emitted from the nanostructure is different in the absence or presence of target. For example, for a fluorescent nanostructure, the associated structure can be a fluorescent quencher molecule or second nanostructure having bound thereto a fluorescent quencher molecule. Also, it can include an acceptor or donor for FRET transfer, etc., or it can modulate through metal-ligand interaction. Thus, one signaling nanostructure of this invention, which is part of a “nanomachine”, is composed of a basic photonic nanostructure, such as a quantum dot, to which a polynucleotide capture probe has been attached. The polynucleotide capture probe is designed so as to hybridize to a specific “target” DNA sequence. Once hybridized, another section of the target DNA sequence is able to hybridize to a secondary oligonucleotide probe (i.e. an “associated structure”) which contains a signal influencing element, such as a quencher group. The term “signal influencing” as used herein refers to e.g., modulating, reflecting, quenching, enhancing, amplifying, tuning or focusing. The proximity of the quencher group to the quantum dot now causes the fluorescent emission of the quantum dot to decrease (dim).

Upon application of a secondary energy input, such as a DC electric field, the quencher group and the quantum dot separate from one another, causing the quantum dot emission to now increase. In the case of a pulsing DC electric field, the specific target bound signaling nanostructure can be made to “blink”, relative to non-target bound signaling nanostructures.

Accordingly, as described above, the “associated structure” is so named, because it is adapted to “associate” with the nanostructure in a signal-influencing manner, which usually means that both the nanostructure and the associated structure bind to the target in close proximity. Thus, the associated structure is assembled to include a target binding region, which usually has a target binding member attached thereto as described above for the nanostructure.

Third Generation Nanostructures

Another aspect of the present invention is a signaling nanostructure that can be oriented such that signal emissions from the nanoconstructs are not random. In one embodiment, the signal may be emitted only from non-quenched regions. Such nanostructures may be aligned in a field which facilitates the ability to look at orthogonal planes of information, i.e. different directions, to better analyze the pulsing output.

In one embodiment of the invention, a signal beam oriented nanostructure is composed of a basic photonic nanostructure containing an asymmetrically positioned nanomechanical element and an associated temporally varying distance dependent interaction element, with the remaining basic nanostructure encompassed with a signal influencing “coating”, such as secondary reflective nanostructures. In this particular embodiment, the basic nanostructure can be a Quantum dot, a fluorescent polymeric nanoparticle, a metallic nanoparticle, or a chromophoric protein complex. The asymmetric positional nanomechanical element and associated temporally varying distance dependent interaction element can be an attached polynucleotide sequence which is complementary to a target DNA sequence, which binds the target sequence in such a fashion as to allow a second polynucleotide sequence containing a quencher group (or FRET donor-acceptor group), which is now so positioned as to either quench (or FRET transfer) to the basic photonic nanostructure.

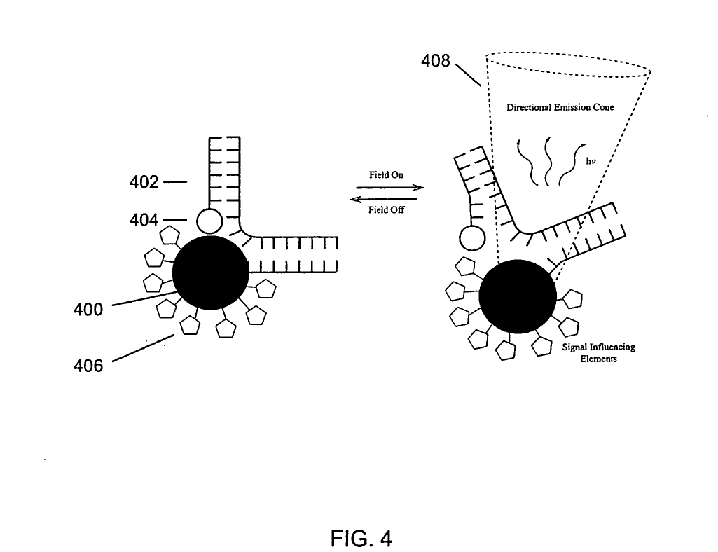

The remaining basic nanostructure surface is now encompassed with signal influencing elements such as signal influencing molecules or nanostructures which can include quencher molecules, other quantum dots, metallic nanoparticles, or other elements which can quench, reflect, enhance or modulate the basic photonic nanostructure. These encompassing nanostructures (molecules) can also be used to incorporate an asymmetry of charge on the overall signaling nanoconstruct, i.e., one side more positive, one side more negative. However, such charges are incorporated in such a fashion (geometry) that they do not cause the signaling photonic nanoconstructs to aggregate by electrostatic interactions. FIG. 4 shows a general diagram of such a signaling photonic nanoconstruct designed for DNA hybridization analysis for single base differences (SNPs, mutations, etc.) in target DNA sequences. When such signaling photonic nanoconstructs are used in homogeneous based hybridization analysis and subjected to input of energy (applied pulsing DC electric field), such constructs will produce an oscillating directional signal which identifies the nature of the target DNA sequence.

Nanomechanical Elements

The nanomachine is further designed with mechanistic properties that allow it, upon the application of energy of appropriate strength and frequency, to produce a detectable and resolvable oscillating signal. In the case of a fluorescent nanostructure/associated structure-quencher combination exposed to a pulsed energy field, this would take the form of a temporal increase and decrease in fluorescent signal (i.e., “blinking”).

Oscillation is facilitated by incorporating a “nanomechanical element” into the system. The term “nanomechanical element” refers to the element (portion, region, member) of the nanomachine that constrains movement of the nanostructure relative to the associated structure (in-plane angular, linear, cylindrical, helical, etc.). Examples are hinges, springs, rotors, etc.

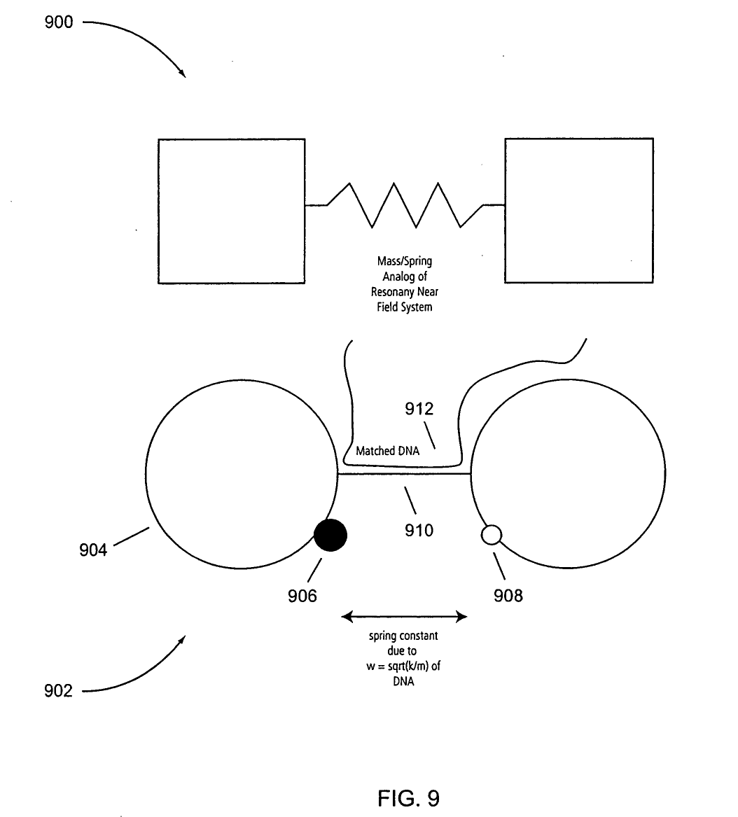

An exemplary embodiment of a nanomachine with a nanomechanical element in the form of a “spring” involves self assembling nanostructures (quantum dots, fluorescent beads, etc.) with signal influencing elements bound thereto (metal nanoparticles) that are further derivatized with probe DNA (target binding elements) that hybridizes with target DNA such that the signaling nanostrucutres are contained within the cleft of the signal influencing elements. Application of free energy (whether electrical, shear, or thermal in the form of solute driven input) to the system will cause the dominant eigenmodes of the system to oscillate, much like a mass/spring system described in simple harmonic motion (as shown in FIG. 9). As it is known that double stranded DNA has far more rigid character than single stranded DNA, mismatches in probe binding should be revealed as frequency differences in the eigenmodes. It is also known that the near field coupling of the metal nanoparticles is strongly distance dependent.

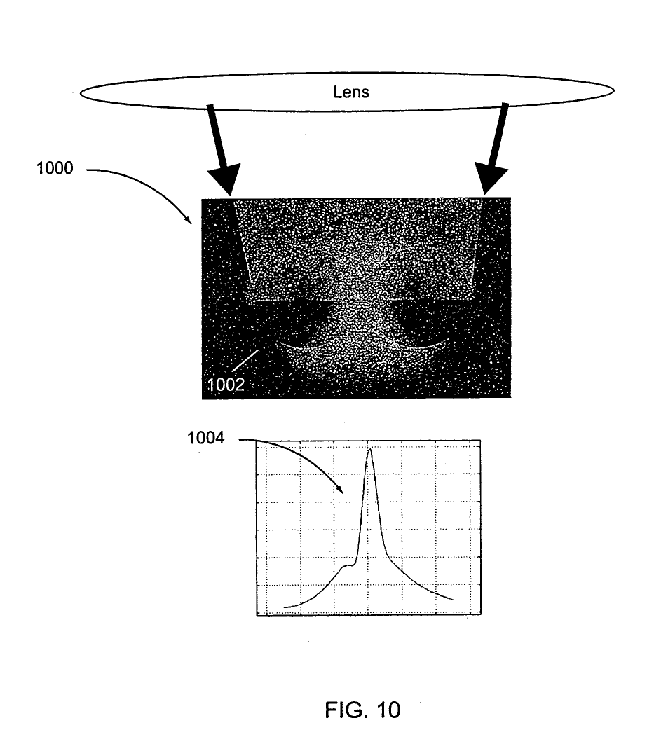

Upwards of three orders of magnitude increase in electric field concentration has been computationally observed within the cleft of the metal particles (FIG. 10), that when modulated by the spring constant of the bound DNA, will result in an optical signal that facilitates detection. FRET pairs could be mounted directly onto the metal particles, or assembled onto the DNA within the cleft and the red shifted peak could be detected. Similarly, a quantum dot could be stationed within the cleft and its amplitude fluctuations could be detected as a result of the near field effect. Because the signal from the nanomachine is based on a distance dependent enhancement of the nanostructures, the amplification is being used differently. FIGS. 9-13 demonstrate different configurations of this embodiment.

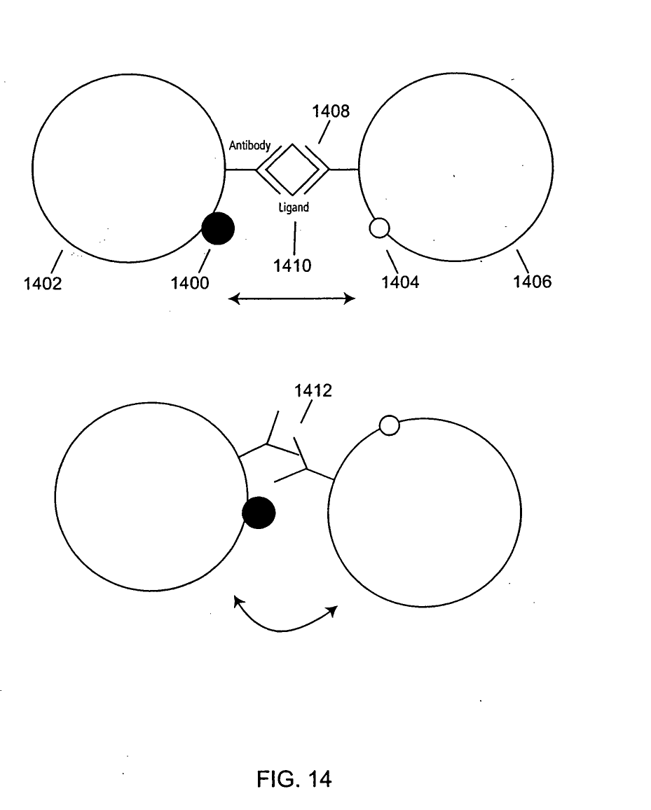

The aforementioned embodiments can also be configured to detect proteins or small molecules, for instance, by replacing the DNA probes with antibodies. It is best explained for the near field effect where a sandwich assay between antibody derivatized metal nanoparticles and a target ligand come together to form the optically amplified, oscillating cleft. In the case of a nonspecifically bound analyte, it is likely that the near field cleft will be sufficiently displaced from the fluorescent group that the amplification effect will be greatly diminished. Furthermore, the eigenmodes of the system will contain more angular momentum than linear momentum, which will drastically change the frequency response of the nanoconstruct. FIGS. 14-17 demonstrate different configurations of this embodiment.

An added benefit of the spring structures is that in certain configurations, gold nanoparticles effectively quench unbound systems, and only those properly assembled systems with a near field amplification surrounding the signaling nanostructure are bright. This lessens the need for high power illumination, thereby reducing scatter and background from other autofluorescent structures. Unlike other traditional near field, or whispering mode sensors designed for high sensitivity (i.e., Vollmer F, Arnold S, Braun D, Teraoka I, Libchaber A. Multiplexed DNA Quantification by Spectroscopic Shift of Two Microsphere Cavities. Biophysical Journal, 851 1974-1979, 2003), the information flow from the nanomachine is a direct result of the dynamic interactions between the target, associated structure, and nanostructure; in particular the information is a result of mechanical (geometrical) changes in the structure rather than simple shifts in the electronic modes of the nanostructure (as in many second generation sensors). The differential effect between bound and unbound excitation of attached nanostructures should enable drastically more sensitive detection of biomolecules.

Input

Energy fields or forces which can be used for input into the nanomachines include macro/microscopic DC/AC electric fields, electrophoretic or dielectrophoretic fields, double layer electric fields (surfaces), capacitance effects (surfaces), optical or photonic energy fields, magnetic fields, pH, thermal energy, ionic strength, fluidic motion or shear and pressure forces. For instance, when immediately juxtaposed to a conductive surface (i.e., within a few Debeye lengths away), electric fields may be used to alter the structures. This is in contrast to electrophoretic fields which are the result of steady state ion gradients in the fluid. Devices which employ electrophoretic fields take great care in eliminating the electrolysis products and pH changes which accompany them. Because of their nanoscale dimensions, these nanomachines can accept energy from electric fields within a few Debeye lengths of a surface.

When designed to have sharp resonant modes, background thermal excitation can be used to drive the nanomachines. By altering the temperature of the surrounding fluid, the average energy input to the system is mediated. In one embodiment, thermal input can be slowly varied when looking for resonant modes in spring structures, or in another embodiment, pulsed to alter hybridization in hinge structures. Temperature oscillations in second generation nanosystems have been achieved upwards of 10 kHz (Braun D, Libchaber A. Lock-in by molecular multiplication. Applied Physics Letters, 83(26): 5554-5556, 2003) by pulsed infrared light onto a microchannel.

Output

Unlike amplified, classical fluorescent microarray systems, low copy number nanoparticle systems pose unique challenges that are quite distinct from perturbations of large populations of fluorescence. When observing single molecule mechanics, it is very unlikely that a single frequency will be the dominant mode of emission from the nanostructure. Thus, our third generation assays present unique challenges in detection.

For instance, in comparison to classical microarray systems where the a priori probability that a test-site region will be detectable is higher than between the test-site regions [i.e., p(a|b)=f(x,y) for microarrays, where the event a is the likelihood of finding fluorescence, and the condition b is equivalent to the statement, “given a particular location in space, such as where the capture DNA was spotted onto the slide,” and f(x,y|test-site)>f(x,y |non test-site)], the third generation systems described herein are generally uniform in their distribution across the sample space [p(a|b)=C, with the constant roughly equivalent to the area (or volume) of the capture region normalized by the concentration of probe]. While the systems described herein are not precluded from being designed with a priori regions for capture, the preferred heterogeneous and homogeneous embodiments which improve rare target kinetics (or for homogeneous assays in general), are largely spread out to minimize the diffusion path length of rare targets.

By repeatedly introducing energy into the system, the probability distributions in the between states can be measured, and downstream detection algorithms (classifiers, Bayesian networks, detection based on sufficient statistics, MAP detection, neural networks, M-ary detection, etc.) can be employed to differentiate the output from the nanomachine. Ideally, the signaling nanoconstruct will oscillate between two maximally separate states as the energy is introduced, and alterations in the driving force mediate the residence time in the stabilized or destabilized state, and the frequency response of matched or mismatched probes can be established. However, we recognize that many nanomachines will exhibit non-ideal behavior, where there is a more stochastic component to the frequency response.

As such, preferred embodiments of the present invention leverage techniques such as parameter estimation, stochastic detection, Bayesian classifiers, neural networks, lock-in amplifiers and such to be able to distinguish molecular kinetics that are vastly different than population behaviors. For instance, in U.S. Pat. No. 6,048,690, the sinusoidal-like behavior that exhibited different amplitudes (but phase matched oscillations) was likely the result of millions of Gaussian distributed binding events overlapping to smooth out the signal. Single molecule dynamics are random in nature, with an applied force (electrical, thermal, etc.) generally affecting the probability of binding such that the average behavior of DNA hybridization is altered, but with the amplitude of oscillation being similar over time. It is the collective dynamics that determine differences in amplitude in high density systems. The ideal nanoscale oscillator would maximize the initial signal to noise ratio, regardless of downstream detection. Furthermore, the ability to properly drive the reversible interactions between the nanostructure and associated structure will largely determine the bandwidth of the response. Therefore, there is a premium on designing systems which have sharp resonant modes in their mechanistic properties.

For instance, in one embodiment, a homogeneous assay where assembled nanomachines are serially processed to flow through a microchannel would interrogate structures as they passed by a detection beam. Nanomachines designed to have resonant modes in the tens of kHz region could be interrogated many thousands of times while drifting in and out of the field of view by using a PMT with a sampling rate in the Mhz range. Temporal signatures from migrating species would likely be decomposed into reduced dimension eigenvectors through preprocessing (multi-taper spectral estimation, other Fourier techniques) followed by a generalized SVD (Kung Sy, Diamantaras K I, Taur J S. Adaptive Principal Component EXtraction (APEX) and Applications. IEEE Transactions on Signal Processing, 42(5): 1202-1217, 1994), mutual information extraction, or Hecht-Nielsen feature attraction (Hecht-Nielsen R, McKenna T, Computational Models for Neuroscience: Human Cortical Information Processing. Springer Verlag, January 2003). These features would then be processed with standard classifiers, Hidden Markov models, or through Antecedent Support hierarchies to facilitate detection.

These techniques are also applied to combined homogeneous/heterogeneous assays where the kinetics of rare target capture are driven in solution by a plurality of nanostructures and associated structures in far excess to the target. Fluidic processing strategies then deposit the captured targets within assembled constructs (through either differential electrical, density, or fluidic drag responses) onto a surface or into a virtual array (i.e., by being held within a dielectrophoretic influence). An image capture device such as a high speed CMOS camera or CCD then interrogates an entire field of nanoparticles in parallel. In this case, the driving signals are likely be in the tens of Hz range to facilitate electrochemical ion gradient steady states after application of fields, and to enable inexpensive cameras to be used.

The method of the present invention also includes application to conventional heterogeneous (microarray or nanoarray) formats in which targets and/or nanostructures are attached to known x,y positions on the array.

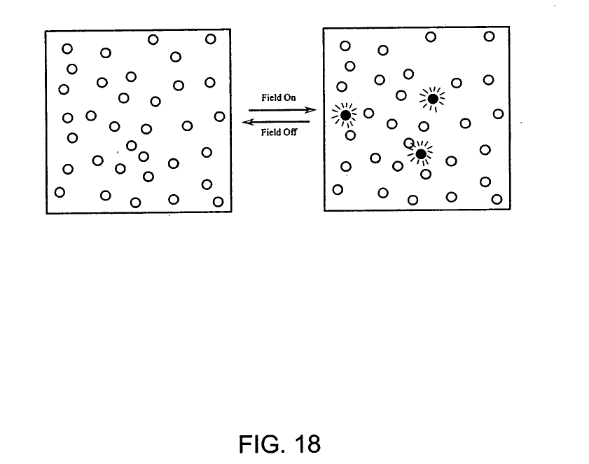

FIG. 18 shows the information flow from a field of nanostructures (circles). Only those nanomachines which are properly assembled demonstrate the signals of interest. The properly assembled nanomachines are represented by the three dark circles in the right pane of the figure. The right pane shows the nanostructures upon the introduction of energy into the system. The left pane shows the nanostructures without introduced energy. Upon repeated introduction of energy into the system, a periodic emission facilitates detection of the specific targets despite the high background signal. Note that in contrast to traditional molecular diagnostic assays, the background of nonspecifically bound nanostructures is far higher than the signal of interest. This is the case in rare target detection, where the probability of a probe binding to a target is far lower than the probability of being nonspecifically captured. Note that endeavors to wash away the nonspecific background are far less important in this configuration. Further embodiments of this technique use two dimensional feature attraction to parse the field into regions of interest (i.e., searching for a region of highly correlated conditional probabilities, coherent emissions, etc.) prior to detection. The aforementioned temporal processing strategies (feature attraction, classification, etc.) are applied to each pixel or region of interest to facilitate detection.

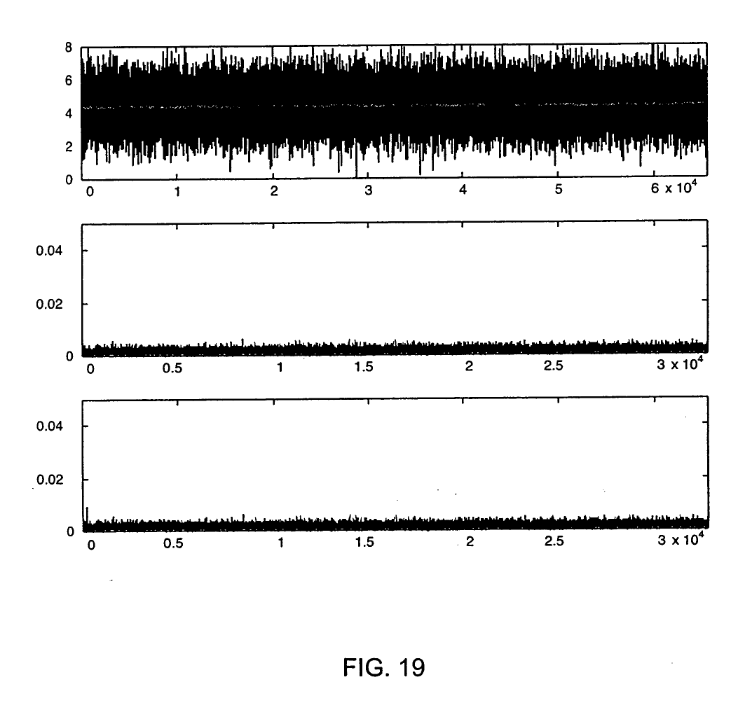

FIG. 19 depicts the results of a simulation demonstrating an oscillatory signal embedded within a very high background of noise (top). The top graph in FIG. 19 is a time based curve of the amplitude of the oscillation, illustrated as the light trace near the center of the noise, which is illustrated as the black region. As shown, the amplitude of the oscillation (the light center trace) is 1% of the noise process. A frequency spectrum plot of the noise process alone is shown in the middle pane. The bottom plot in FIG. 19 illustrates a frequency spectrum plot of the noise and the embedded signal. As illustrated in the bottom plot, when observed for a sufficient amount of time, the signal is clearly evident as a peak amidst the noise (the peak near the origin, on the left side). A traditional assay would measure the signal-to-noise ratio in this situation as 1:100 (for a 1% signal). By adding in a spatially independent, temporally varying signal, the measured signal to noise is roughly 3:1. Accordingly, the peak height is three times the noise. This ratio can be increased by observing the system for a longer time, or by increasing the speed of oscillation and observing for the same amount of time.

Temporally varying, spatially independent signals are inherently more sensitive than their static counterparts. FIG. 19 demonstrates the benefits of the temporal processing strategy. Fourier methods, and similar signal processing strategies known to those skilled in the art will recognize that periodic signals can be substantially lower than the background noise yet are still extracted when properly processed. The longer one observes the oscillation, the better the extraction. This is in contrast to existing molecular diagnostic assays which generally observe a single time point in order to make a detection decision. For instance, as demonstrated below, the probability of detection P_{D} of a signal given a collection of samples x of a signal embedded within Gaussian noise=N(a,σ²), as n, the number of samples increases, the limit of P_{D} goes to 1.

$${P}_{D} = \frac{1}{\sqrt{2 π {σ}^{2} / n}} {\int }_{t}^{∞} {ⅇ}^{- \frac{{n ( x - 1 )}^{2}}{2 {σ}^{2}}} ⅆ x = Q ( \frac{{n}^{1 / 2} ( t - 1 )}{σ} )$$

Similar arguments for detection can be made for signals embedded within non-Gaussian noise.

Furthermore, because the information flow from the nanomachines is intimately tied to the reversible mechanical interactions, the ability to orient and align signals from the nanostructures (and the coupled signal influencing elements therein), increase our ability to look at orthogonal planes of information, i.e. different directions, to better analyze the periodic output. In many ways, this is analogous to the ways in which pulsars are picked out from a field of stars in the sky. In this manner, vector valued data demonstrating maximum orthogonality between dimensions can be used to assist in detection.

The method of the present invention includes a signal processing step, which is, in some sense, a “mathematical washing step.” This process entails a mathematical separation of the true signal from background which takes advantage of the oscillating signal emitted by the optically transducing nanoconstruct. For example, in U.S. Pat. No. 6,048,690, the hybridization assay (heterogeneous format) includes many washing steps (20 mM NaPhosphate, pH 7.2, at room temperature, 3 times for 10 minutes each wash; col 21 lines 22-23). The present invention allows for the same assay to be performed in a heterogeneous or homogeneous format where the “mathematical washing” step replaces physical washing, and the speed of the overall process is increased significantly. The temporal information content from the target bound nanoparticle probes allows one to discriminate between specifically and nonspecifically bound particles, without having to remove the majority of unbound or nonspecifically bound probes.

The ability to detect spatially independent nanomachines also endows the sample preparation steps in assays with new possibilities. For instance, in the assays described herein (either homogeneous or heterogeneous), where nanostrucutres and associated structures are flowed into the sample in great excess to drive kinetics, complex biological samples can be automatically analyzed by electroporating or sonicating contents within a channel, then the targets can be concentrated with electric fields (i.e. turning a field positive for a period of time until the bound targets and nanomachines migrate into a smaller volume, further driving kinetics) followed by revervising the field and expanding the volume (which drives the thermodynamic process towards the global minima, in order to perturb nonspecifics; but also to drive capture of rare targets, because even if in great excess, complex biological samples may overwhelm the target in terms of binding sites). Because the nanomachines are spatially independent, these concentration/expansion steps can be performed repeatedly in solution before the nanomachines are separated, either with dielectrophoresis or simpler fluidic processing strategies (separating by weight, density, etc.) to collect the bound targets onto an array or virtual array for signal processing.

As discussed, the ability to interrogate time varying spatially independent structures allows a myriad of heretofore unavailable assays. Considerable improvement in existing assays can also be realized. Prime examples are use in PAP smears and in situ hybridization tests.

Similarly, because the nanomachines are designed to be spatially independent, they can be placed in a transverse electrophoretic field where two macroelectrodes spaced far apart drive the energy input into the system. This would be preferable for applications such as in situ hybridization techniques, because very high voltages could be applied (as in submarine gel formats). The nanomachines described herein would significantly improve the FISH platform by eliminating the need for the many steps required to reduce background. A fluorescent in situ hybridization assay using traditional fluorophores suggests the following procedure:
1. Fixation of material on slide (ethanol precipitation or formaldehyde cross linking)
2. Pretreatment of specimen (to reduce background)
3. Prehybridization (incubation with hybridization solution minus probe)
4. Denaturation of probe and target
5. Determination of hybridization temperature
6. Determination of hybridization pH
7. Determination of hybridization solution composition
8. Determination of probe concentration
9. Hybridization (slow kinetics, hours)
10. Post-Hybridization stringency washes
11. Determination of hybridization specificity controls
12. Detection

The total time required for these steps can reach many hours of continual attention by a dedicated technician. The procedure becomes more difficult and requires more skill when multiplexing probes. In contrast, the current invention would enable the following protocol:
1. Fixation of material on slide (ethanol precipitation or formaldehyde cross linking)
2. Hybridization (fast kinetics, seconds to minutes)
3. Rinse
4. Detection

Since the majority of the steps required to perform FISH are explicitly designed to reduce background noise, we can eliminate virtually all of them by using temporal signals to distinguish between specifically and nonspecifically bound probes. Moreover, FISH manuals recommend stringent hybridization conditions, i.e., pushing the hybridization to the brink of stability even during the initial phase and increasing the time required for traditional FISH to incubate. The nanomachine invention allows us to drive the kinetics of nucleation in favor of hybridization by flooding the system with probes in non-stringent conditions. With minimal rinsing in stringency buffer, we would then be ready to perform detection.

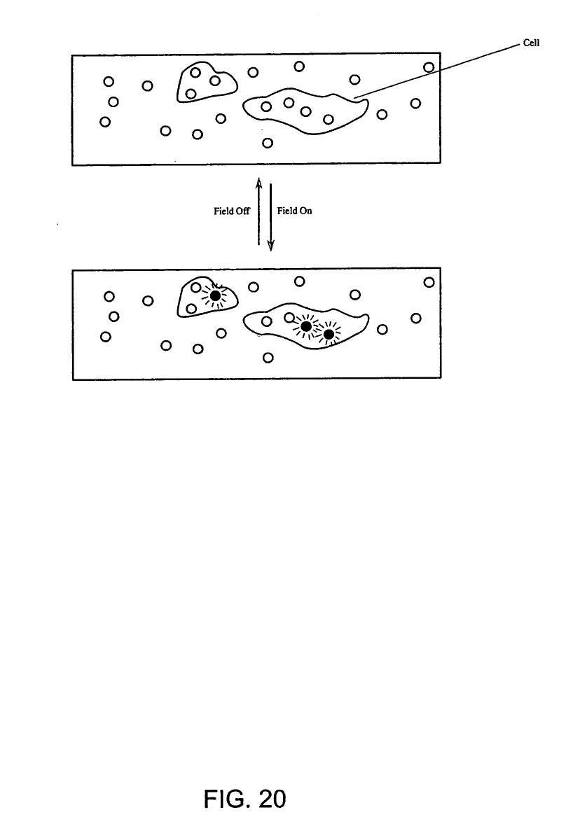

As shown in FIG. 20, the information flow from a field of nanomachines within a fluorescent in situ hybridization assay (FISH) is greatly enhanced. Only those nanomachines that are properly assembled demonstrate the signals of interest. The properly assembled nanomachines are represented by the dark circles within the cellular boundaries.

The signal processing strategies herein are optimized for systems exhibiting a reversible interaction between a nanostructure, associated structure and target, regardless of the interaction type. Because of this, the current invention is largely independent of the final detection methodology; gathering information about induced reversible mechanical alterations can be used to facilitate fluorescent detection, SERS or Raman peak shifts, or in the case of a whispering mode gallery sensor, the transition between spectral peaks. The current invention can transduce energy into electrochemical changes (i.e., oscillations in redox reactions atop a measuring electrode), impedance changes (as in voltammetry based detection), or even STM type measurements where individual nanostructures are juxtaposed between the tips of nanoelectrodes and tunneling currents are measured as a result of the induced mechanical properties of the assembled nanomachine. It is the assembly of the higher order structures which allow information about the target to be passed along to the macroscale. For instance, one could envision a field of nanoelectrodes which are used in the combined homogeneous heterogeneous capture assay, wherein assembled nanomachines are captured between two nanoelectrodes. Because it doesn't matter which electrode the machines are trapped between, the temporally varying, spatially independent signal will identify both the presence and specificity of binding.

It is also within the scope of this invention to carry out multiplex detection. Multiplex detection of different target sequences in the same sample can be achieved by using basic signaling nanostructures which produce a different wavelength of fluorescent emission (blue, green, orange, red, etc.). In this case, the detection system would not only be designed to pick up oscillation in target bound signaling nanostructures, but would also observe the sample at different emission wavelengths; i.e. a first target sequence might appear as green blinking signals and the second target sequence might appear as red blinking signals. Heterogeneous multiplexing may also be carried out to take advantage of the spatially independent nature of the nanomachines. By splitting a linear fluidic feed into parallel channels, each with a uniformly distributed lawn of nanostructures (with uniformly distributed colors), multiplexing can be carried across many targets even with the same basic color scheme (by having different lawns in each parallel channel).

Additionally, it is within the scope of this invention to use the nanomachines in classical microarray formats. The signaling nanomachines should greatly benefit the specificity, speed and sensitivity when placed into predetermined (x,y) coordinates. Because of the ability to substantially eliminate PCR amplification and washing steps, the kinetics of nanomachine microarrays should be significantly faster. Moreover, the benefits of the signal processing strategies described herein greatly improve the sensitivity of standard arrays. Therefore, the benefits of the invention are widely applicable to classical formats as well as the novel assays described herein.

Target Detection

The embodiments described herein are useful for detecting any molecular target. More particularly, the present invention concerns the detection of a member of a molecular binding pair—that is, two molecules, usually different that, through chemical or physical means, specifically bind to one another. Therefore, in addition to antigen and antibody specific binding pairs of common immunoassays, other specific binding pairs can include biotin and avidin, carbohydrates and lectins, complementary nucleotide sequences, effector and receptor molecules, cofactors and enzymes, drugs and receptors, enzyme inhibitors and enzymes, and the like. Furthermore, binding pairs can include members that are analogs of the original specific binding members, for example, an analyte-analog.

Further, the present methods are useful for the detection of specific binding events such as DNA hybridization, immunochemical reactions, protein/ligand binding, drug/receptor binding and metal/ligand binding. Accordingly, suitable targets include, for example, proteins, small molecules, peptides, receptors, cells, viruses, nucleic acids, hormones, antibodies, antigens, enzymes, substrates, ligands, small molecules and the like.

It should be understood that as used herein, the term “target” refers not only to unknown targets in a sample, such as a clinical sample, but also refers to any member of a molecular binding pair. Accordingly, the target can be any molecular structure, whether singular or part of a larger macromolecular structure, and thus the present invention is useful for imparting any known member of a molecular binding pair with a detectable signal (which is sometimes referred to as labeling).

By way of example, the target may be a nucleic acid, which intends any polymeric nucleotide (i.e. “oligonucleotide” or “polynucleotide”), which in the intact natural state can have about 10 to 500,000 or more nucleotides and in an isolated state can have about 20 to 100,000 or more nucleotides, usually about 100 to 20,000 nucleotides, and more frequently 200 to 10,000 nucleotides. For example, the assay can be adapted to detect any target nucleic acid with a determined nucleic acid sequence that is characteristic of a cell type, cell morphology, pathology, bacteria, microbe, virus, etc.

The term “nucleic acids” includes duplex DNA, single-stranded DNA, RNA in any form, including triplex, duplex or single-stranded RNA, anti-sense DNA or RNA, polynucleotides, oligonucleotides, single nucleotides, chimeras, and derivatives and analogues thereof. It is intended that where DNA is exemplified herein, other types of nucleic acids would also be suitable. Nucleic acids may be composed of the well-known deoxyribonucleotides and ribonucleotides composed of the bases adenosine, cytosine, guanine, thymidine, and uridine, or may be composed of analogues or derivatives of these bases. As well, various other oligonucleotide derivatives with non-phosphate backbones or phosphate-derivative backbones may be used. For example, because normal phosphodiester oligonucleotides (referred to as PO oligonucleotides) are sensitive to DNA- and RNA-specific nucleases, oligonucleotides resistant to cleavage, such as those in which the phosphate group has been altered to a phosphotriester, methylphosphonate, or phosphorothioate may be used (see U.S. Pat. No. 5,218,088).

The nucleic acid target can be naturally occurring and assayed with minimal further purification from a biological sample, or it may be isolated from the natural state, particularly those having a large number of nucleotides, frequently resulting in fragmentation, which in turn results in the target consisting of a size-heterogeneous population of nucleic acids.

The nucleic acid targets include nucleic acids from any source in purified or unpurified form including DNA (dsDNA and ssDNA) and RNA, including t-RNA, m-RNA, r-RNA, mitochondrial DNA and RNA, chloroplast DNA and RNA, DNA-RNA hybrids, or mixtures thereof, genes, chromosomes, plasmids, the genomes of biological material such as microorganisms, e.g., bacteria, yeasts, viruses, viroids, molds, fungi, plants, animals, humans, and fragments thereof, and the like. In one embodiment, the target is a double stranded DNA (dsDNA) or a single stranded DNA (ssDNA). The target can be obtained from various biological material by procedures well known in the art.

The target may also be recognizable by an antibody, in which case the target is any epitope or antigen, or any immunoreactive molecule, including antigen fragments, antibodies and antibody fragments (to which anti-immunoglobulin antibodies bind), both monoclonal and polyclonal, and complexes thereof, including those formed by recombinant DNA molecules. The term “hapten”, as used herein, refers to a partial antigen or non-protein binding member which is capable of binding to an antibody, but which is not capable of eliciting antibody formation unless coupled to a carrier protein.

As previously mentioned the target may be present in an industrial or clinical “test sample”, which includes biological samples that can be tested by the methods of the present invention described herein and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas and the like, biological fluids such as cell culture supernatants, fixed tissue specimens and fixed cell specimens. Any substance which can be diluted and tested with the assay formats described in the present invention are contemplated to be within the scope of the present invention.

Applications

The nanomachines described herein are useful in any setting or application that is facilitated by enhanced detection, and in particular in those application where it is useful to facilitate the flow of information from the molecular or nanoscale to the macroscale level. Accordingly, the nanomachines of the present invention are useful to carry out biosensing, molecular biological and molecular diagnostic analyses including proteomics, genomics, drug screening/identification, genotyping, gene expression, DNA diagnostics (cancer, genetic diseases, infectious diseases), infectious agent detection, bioterror aent detection, and for human identification and forensic applications.

By way of example, the nanomachines of the present invention are useful for genotyping single point mutations, single nucleotide polymorphisms (SNPs) or short tandem repeats (STRs) in the same manner as known assays, such as a plasma based assay, Taqman, restriction digestion of PCR products, calorimetric mini-sequencing assay, radioactive labeled based solid-phase mini sequencing technique, allele-specific oligonucleotide (ASO), and single strand conformation polymorphism (SSCP).

The nanomachines are also useful for nanophotonic and nanoelectronic information transfer applications, as well as for computational or data storage applications.

### Exemplary Embodiments

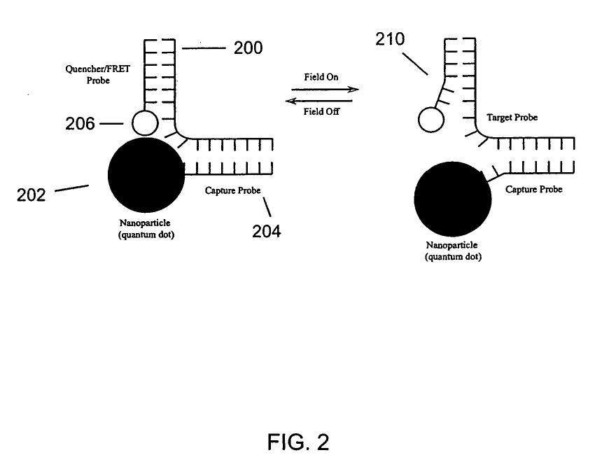

In the discussion that follows, FIGS. 2 to 8 are referred to throughout. These Figures depict alternative embodiments of the present invention as further described below. As shown in FIG. 2, the exemplary target is a nucleic acid 200 (the L-shaped structure) and the exemplary nanostructure 202 is a nanoparticle, such as a quantum dot (black circle, Nanoparticle (quantum dot)) with an oligonucleotide attached thereto 204 (Capture Probe) that binds to the target. Also shown in FIG. 2, the exemplary associated structure 206 is a fluorescent quencher (open circle) with an oligonucleotide attached thereto (Quencher/FRET Probe) that binds to the target in proximity to the binding site of the oligonucleotide attached to the nanostructure. Other elements in the Figures are described in the following discussion.

When such nanomachines are used in hybridization analysis (homogeneous, heterogeneous, or serial homogeneous/heterogeneous) and subjected to input of energy (applied pulsing DC electric field, thermal excitation, shear stress from a fluid, magnetic input, etc.), they produce an oscillating signal that identifies the nature of the target DNA sequence. However, it should be well understood that these nanomachines can easily be adapted for use in other molecular binding assays as described elsewhere herein.

FIG. 2: In one embodiment of the invention, a nanomachine is composed of a basic photonic nanostructure 202, one or more nanomechanical elements, and an associated structure 206 (Quencher/FRET Probe). In this particular embodiment, the basic nanostructure can be a Quantum dot, a fluorescent polymeric nanoparticle, a metallic nanoparticle, a chromophoric protein complex, etc., with a capture probe attached thereto as shown, that is complementary to a target DNA sequence. The associated structure 206 can be a polynucleotide sequence which is complementary to the same target DNA sequence having a signal influencing moiety attached thereto such as a quencher. When bound to the target as shown, the associated structure is now positioned so as to quench, enhance, or modulate the basic photonic nanostructure, thereby creating a temporally varying, distance dependent interaction.

In this example, a hinge 210 functions as the nanomechanical element (shown as the bend in the oligonucleotide of the Quencher/FRET Probe when the field is on), and is designed into the associated structure 206 by destabilizing the affinities of the lower end of the probe (relative to the upper end) and minimizing the interaction between the quencher and nanoparticle. Mismatches within this hinge region would maximally influence the dynamics of the interaction. The mismatches are preferably positioned within 1 to 15 base pairs (preferably 1 to 5) from the quencher, or within 5 nm. When subjected to input of energy (applied pulsing DC electric field, staircase DC field, thermal excitation, shear stress from a fluid, magnetic input, etc.), such constructs will produce a periodic signal which identifies the nature of the target DNA sequence.

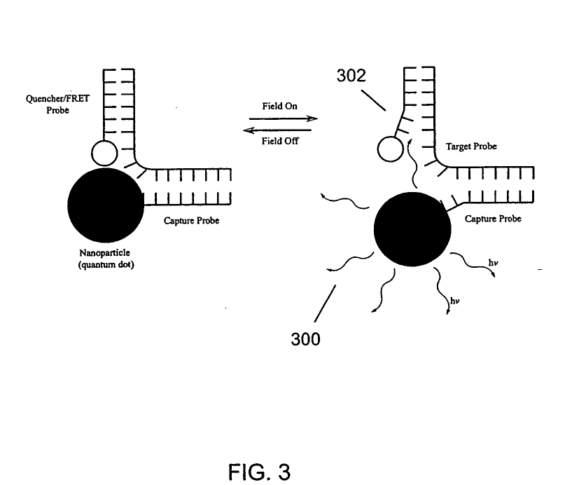

FIG. 3: In another embodiment of the invention, the nanomachine is as described for FIG. 2 above. While the light can be emitted as a spherical wave 300 in all directions (as depicted by wavy lines directed outward from the nanoparticle), fields (i.e., electrophoretic or dielectrophoretic) can be used to orient the particle in such a way that the associated structure is preferably aligned to maximize signal fluctuations, and to interrogate the structure in orthogonal planes. A hinge 302 is also included as described above.

FIG. 4: In another embodiment of the invention, a higher order signaling nanoconstruct is composed of a basic photonic nanostructure 400 and an associated structure 404 with a hinge 402 as described above for FIG. 2. As shown, a region of the nanostructucture 400 (i.e., the signal enhancing region) is encompassed by a plurality of signal influencing elements 406. The signal influencing elements 406 (shown as pentagonal structures) can include secondary nanostructures, quencher molecules, other quantum dots, metallic nanoparticles, or other moieties which can quench, reflect, enhance or modulate the basic photonic nanostructure. The signal influencing elements can also be used to incorporate an asymmetry of charge on the overall signaling nanoconstruct, i.e., making one side more positive and one side more negative. However, such charges are incorporated in such a fashion (geometry) that they do not cause the signaling nanoconstructs to aggregate by electrostatic interactions. FIG. 4 shows a general diagram of such a nanomachine designed for DNA hybridization analysis for single base differences (SNPs, mutations, etc.) in target DNA sequences. When these higher order signaling nanoconstructs are subjected to input of energy, they produce an oscillating directional signal 408, which is depicted as a Directional Emission Cone.

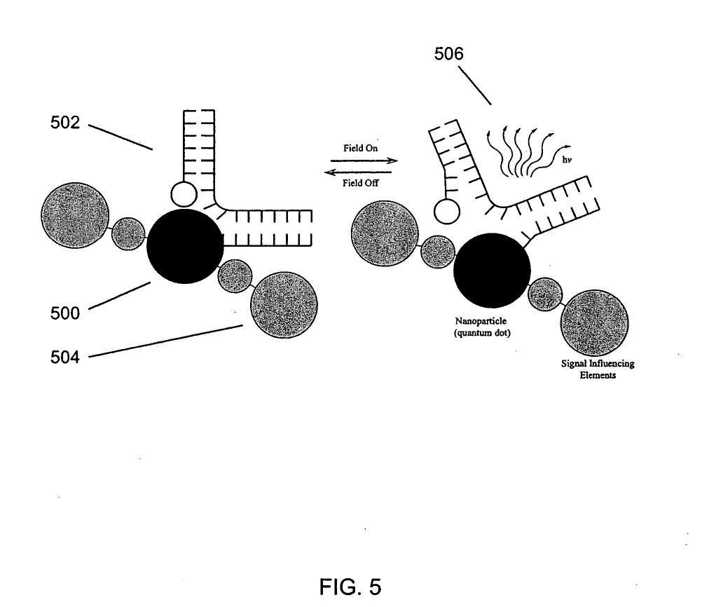

FIG. 5: In yet another embodiment of the invention, a different higher order signaling nanoconstruct is composed of a basic photonic nanostructure 500 and an associated structure with a hinge 402 as described above for FIG. 2. In this embodiment, the signal influencing elements 504 are represented by a metallic bead complex in the form of a nanolens. The metallic beads serve to create a near field excitation center which dramatically enhances the output from the nanoparticle, as shown by the wavy lines directed outward. Each half of the nanolens is shown with a pair of two self-similar metallic beads (grey circles), but it is understood that these lenses can be constructed with one or more metallic particles. As described above for FIG. 4, when these higher order signaling nanoconstructs are subjected to input of energy, they produce an oscillating directional signal.

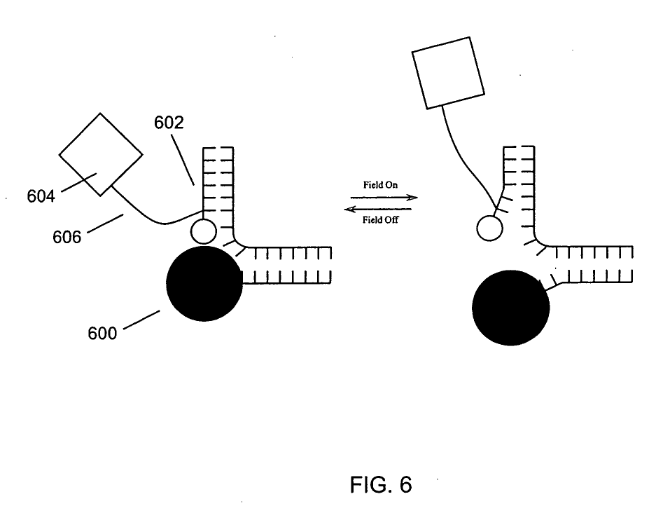

FIG. 6: In another embodiment of the invention, the nanomachine is composed of a nanostructure 600 and an associated structure 602 as described for FIG. 2 above. Also embedded into the associated structure is an interaction amplifying element 604 (open box). The interaction amplifying element helps to displace the associated structure from the nanoparticle by providing tension along a linker molecule 606 (between the open box and the probe). The interaction amplifying element 604 can exhibit a preferential charge, a fluidic drag, or a magnetic moment or any combination of these to enable amplification of the field effect on the nanomachine. FIG. 6 demonstrates this general construct.

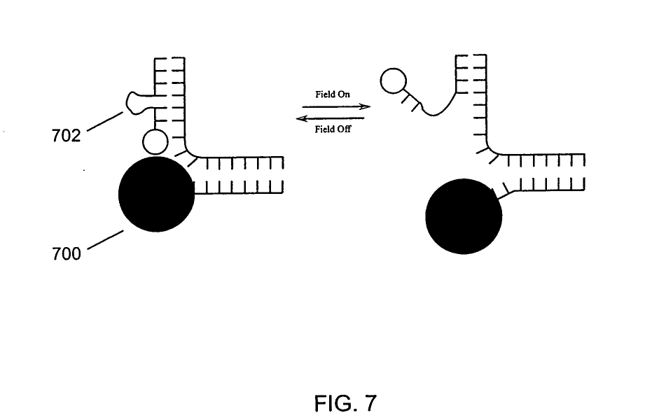

FIG. 7: In another embodiment of the invention, the nanomachine is composed of a nanostructure 700 and associated structure as described above for FIG. 2. One modification in this embodiment is that an interaction amplifying element 702 in the form of a long linker (the loop structure on the left, which opens upon application of energy) will provide a greater displacement of the quencher during oscillation so as to increase the signal change in time. In this sense, the interaction amplifying element enhances the nanomechanical element (hinge).

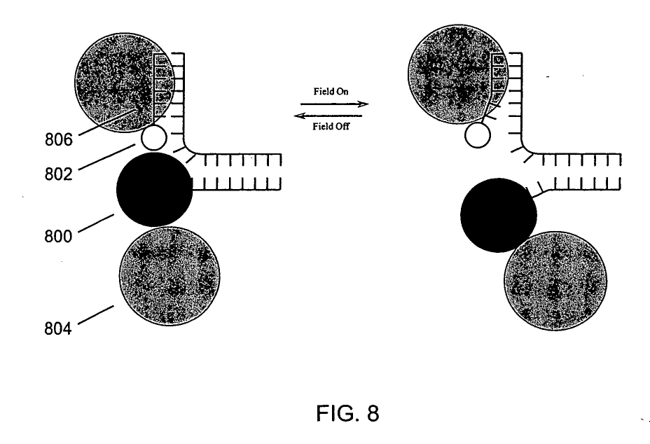

FIG. 8: In another embodiment of the invention, the nanomachine is composed of a higher order signaling nanoconstruct composed of a basic photonic nanostructure 800 and an associated structure 802 as described for FIG. 2. In this embodiment, the nanoconstruct consists of the basic nanostructure 800 having attached thereto a signal influencing element 804. The associated structure 802 also has attached thereto a signal influencing element 806. In this embodiment, the signal influencing elements are represented by metallic beads. The metallic beads serve to create a distance dependent near field excitation center which dramatically enhances the output from the nanostructure in response to the oscillation of the associated structure. Each half of the nanolens is shown with a single metallic bead (grey circles), but it is understood that these lenses can be constructed with a one or more metallic particles.

FIG. 9. This figure relates the mechanistic analog 900 (top half) of a mass/spring system to a linear nanomechanical element 902 (bottom half). The signal influencing elements 904, which can be metallic nanoparticles, etc., also act as a substrate to which the nanostructure 906 is bound and the associated nanostructure 908 assembles. The DNA 910 between the the plasmonic beads acts like a spring between the signal influencing elements. This is in contrast to the hinge structures shown earlier. As shown, the DNA binds to the target 912, which is labeled “Matched DNA”. While the mass/spring depiction 900 is clearly an oversimplification, the nanomachine should have a dominant eigenmode which is similar to the linear mass/spring system.

FIG. 10: This figure depicts the results from a simulation showing the electric field confinement and enhancement between two 50 nm gold nanoparticles 1002 excited by a focused illumination source. These beads are analogous to the metallic beads shown in FIG. 9 (904). Also plotted below the figure is the field cross-section of the electric field enhancement 1004. This representation demonstrates the results from a finite-element model of the electric field increase upon excitation from a plane wave source orthogonal to the long axis of the nanomachine.

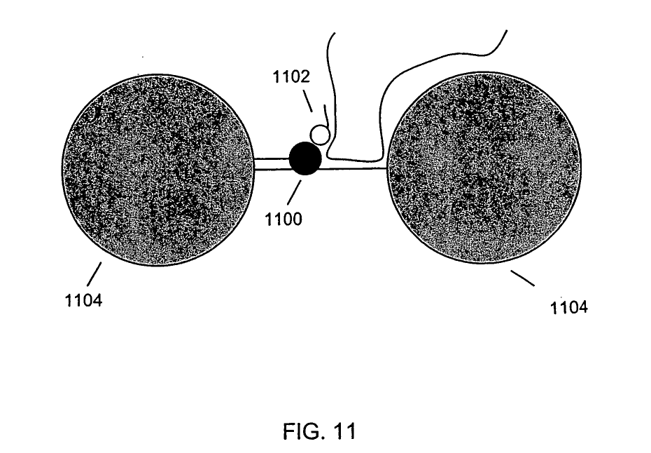

FIG. 11: In another embodiment of the invention, a nanomachine is composed of a basic photonic nanostructure 1100 and an associated structure 1102 (small open circle with “tail”) as described above for FIG. 2, and two nanomechanical elements. In this embodiment, the basic nanostructure 1100 acts as a resonant near field cavity combined with a fluorescent center. One modification from the previously described embodiments is that the surface of the basic nanostructure 1100 is modified by two signal influencing elements 1104 attached thereto. In this embodiment, the signal influencing elements 1104 are represented by metallic beads. The signal influencing elements serve to create a distance dependent near field excitation center which dramatically enhances the output from the nanoparticle in response to the oscillation of the associated structure. Accordingly, this embodiment is an example of a nanomachine with two nanomechanical elements built in, i.e. both a hinge and a spring (the DNA that stretches from one signal influencing element 1104 to the other creates a substantially rigid double stranded structure that exhibits linear springlike behavior along the central axes of the DNA to form the nanomechanical element as shown in FIG. 9). This configuration assists in maximizing orthogonality in the detected signal.

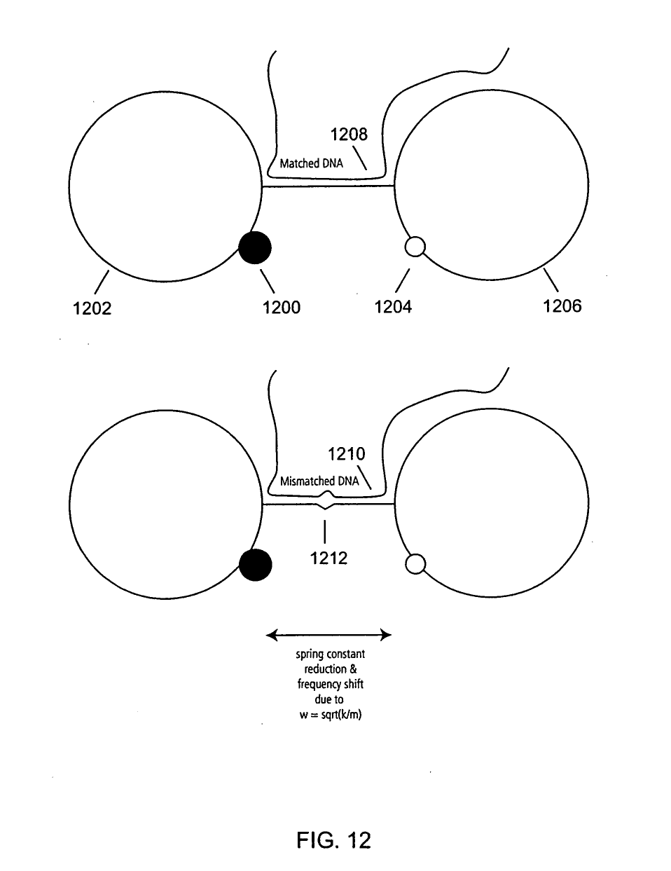

FIG. 12: In another embodiment of the invention, a nanomachine is composed of a basic photonic nanostructure 1200 with a nanomechanical element and an associated structure 1204. The basic photonic nanostructure (as described for FIG. 2) has one signal influencing element 1202 attached thereto in the signal influencing region (in proximity to the signal influencing element), and a second signal influencing element attached to an associated structure 1204. As depicted, the signal influencing elements act as a resonant near field cavity. In this particular embodiment, the signal influencing elements are comprised of metallic nanoparticles, preferably in the 10-50 nm range. The nanostructure and associated structure are located within the cleft of the near field cavity and produce a FRET response when coupled. Hybridization of the target (either Matched DNA 1208 or mismatched DNA 1210) creates a substantially rigid double stranded structure, which exhibits linear springlike behavior along the central axes of the DNA to form the nanomechanical element as shown in FIG. 9. The signal influencing elements serve to create a distance dependent, near field excitation center which dramatically enhances the output from the nanostructure 1200 in response to the oscillation of the assembled mass/spring system.

Because the near field excitation decreases almost exponentially with distance, small deflections in the cavity will substantially alter the signal from the nanoparticles. Furthermore, if nonspecifically bound (i.e. mismatched) DNA happens to hybridize to the nanomachine, the mismatched bases 1212 will exhibit more single strand character than the corresponding matched double strand. Frequency differences in the mechanical oscillation between matched and mismatched DNA will be mediated by this change in double stranded character, because the spring constant of the oscillator will be altered by the differences in DNA. The nanolens is shown with a pair of self-similar metallic beads, but it is understood that these lenses can be constructed with one or more metallic particles. When the nanomachine is subjected to input of energy (as described above), it will produce an oscillating signal that identifies the nature of the target DNA sequence. In this embodiment, it is also understood that the system can be driven by the background thermal energy of the solvent. Structures can be designed to have sharp resonant eigenmodes to maximize coupling at a given temperature, by tuning the mass of the metallic beads. These structures will likely produce substantial oscillations in the kHz-MHz range.

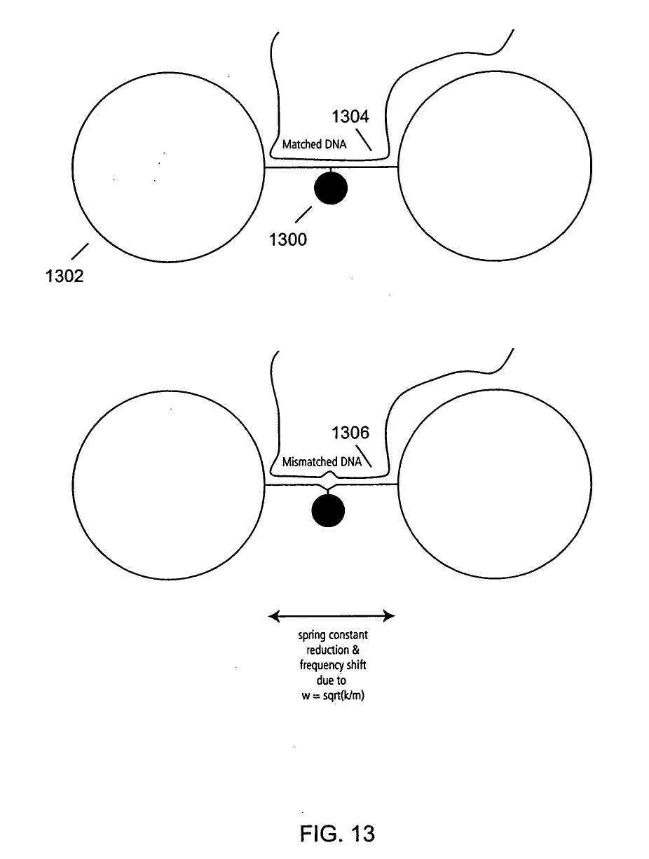

FIG. 13: In another embodiment of the invention, a nanomachine is composed of a basic photonic nanostructure 1300 with a nanomechanical element and an associated structure 1302 (the two metallic beads connected by ssDNA), with the associated nanostructure 1302 acting as a resonant near field cavity (as opposed to the function of the signal enhancing elements described in prior embodiments). In this particular embodiment, the associated nanostructure is comprised of two metallic nanoparticles, preferably in the 10-100 nm range. The basic photonic nanostructure 1300 is as described above for FIG. 2. In this embodiment, the basic photonic nanostructure 1300 is attached within the cleft of the near field cavity. Hybridization of the target will create a substantially rigid double stranded structure (shown as the double lines between the metallic beads), which exhibits linear springlike behavior along the central axes of the DNA as described above for FIG. 12. IN addition, the nanomachine will function as described above for FIG. 12. The metallic beads serve to create a distance dependent, near field excitation center which dramatically enhances the output from the photonically active nanoparticles in response to the oscillation of the assembled mass/spring system. Other properties of the nanomachine are as described above for FIG. 13.

FIG. 14: In another embodiment of the invention, a nanomachine is composed of a basic photonic nanostructure 1400 with a nanomechanical element and an associated structure 1404. The basic photonic nanostructure (as described for FIG. 2) has one signal influencing element 1402 attached thereto in the signal influencing region (in proximity to the signal influencing element), and a second signal influencing element 1406 attached to an associated structure 1404. As depicted, the signal influencing elements act as described for FIG. 12. In this embodiment, the signal influencing elements, 1402 and 1406, are functionalized through the attachment of antibodies 1408 (i.e. target binding elements). The nanostructure and associated structure produce a FRET response when coupled as described for FIG. 12. Binding of the target ligand (open diamond) creates a substantially rigid structure formed by the antigen/antibody complex, which exhibits linear springlike behavior along the central axes of the complex. Other features of this embodiment are similar to those described for FIG. 12 above. Nonspecifically bound complexes 1412 (including other nanostructures or associated structures) as shown in the bottom half of the Figure will contain different eigenmodes than specifically bound nanomachines. As they lack the properly constructed nanomechanical spring, nonspecific nanomachines will produce distinct frequency spectra from specifically bound structures. Furthermore, inefficient plasma coupling between nonspecifically bound complexes 1412 reduces the photonic amplification interaction. Along with the embodiment depicted in FIG. 12, this embodiment shows a general diagram of a nanomachine designed for small molecule, peptide or protein detection.

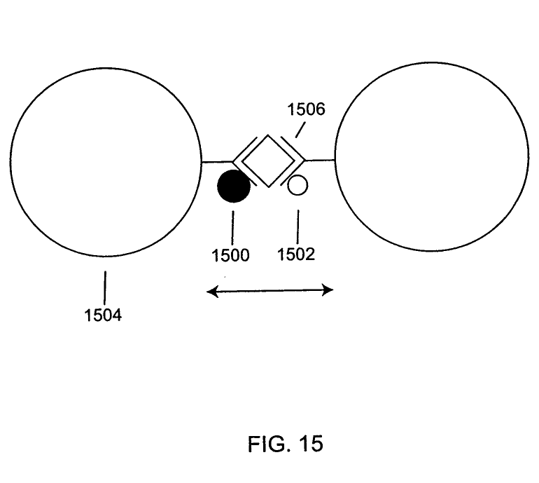

FIG. 15: In another embodiment of the invention, the nanomachine is as described for FIG. 14 above, with the nanostructure 1500 and associated structure 1502 bound to their signal influencing elements 1504 through antibodies 1506.

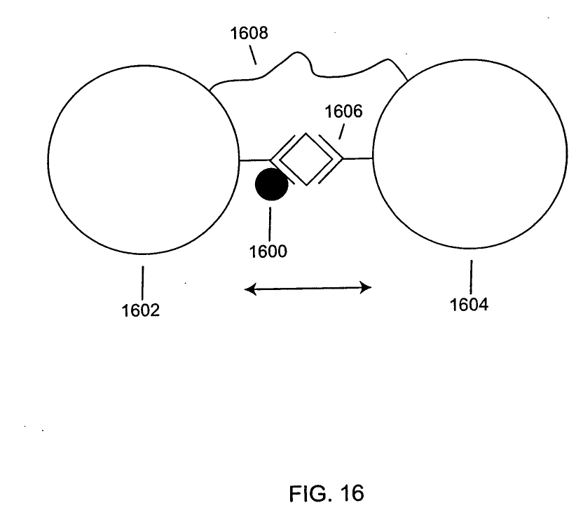

FIG. 16: In another embodiment of the invention, a nanomachine is composed of a basic photonic nanostructure 1600 with a nanomechanical element and an associated structure 1604. The basic photonic nanostructure 1600 (as described for FIG. 2) is attached to an antibody, which in turn is attached to a signal influencing element 1602. The signal influencing element 1602 is functionalized with an antibody as described for FIG. 14. The signal influencing element 1602 and the associated structure 1604 act as a resonant near field cavity. In this particular embodiment, the signal influencing element 1602 and the associated structure 1604 are metallic nanoparticles, preferably in the 10-100 nm range.

In this embodiment, the signal influencing element 1602 and the associated structure 1604 are also attached by a nonbinding, flexible chemical linker, i.e., a tethering structure 1608, shown as the wavy line at the top of the Figure. When unbound, the near field cavity will be largely randomly oriented. Upon binding a target, the cavity will align to produce the now familiar nanomechanical spring. The flexible chemical linker serves to accelerate the sandwich assay kinetics, such that the target need only diffuse to a single nanoparticle complex. The tethering structure can also act as a signal influencing properties. For example, by tuning the osmotic pressure of the sidegroups of the tether, one can alter the eigenmodes of the system because the rigidity of the tether contributes to the mechanical properties of the nanomachine. Branched chain polyalkylene oxides such as polyethylene glycols (PEGs), hydrophilic polymers, amino acid chains, glycosaminoglycan (GAG) chains, etc., can be employed as mechanically tunable tethers. Moreover, when using fatty acids as tethers between signal influencing elements, one may tune the mechanical response of the nanomachine through energy input. For example, altering the temperature of the solvent will mediate the rigidity of the fatty acid chain.

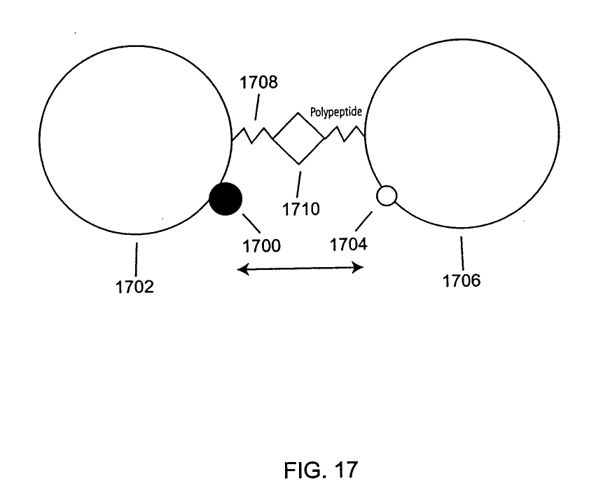

FIG. 17: In another embodiment of the invention, a nanomachine is composed of a basic photonic nanostructure 1700 with a single attached signal influencing element 1702, one or more nanomechanical elements and an associated structure 1704, also with a single attached signal influencing element 1706. Other features of this nanomachine are as described above for FIG. 14. However, in contrast to FIG. 14, the signal influencing elements 1702 and 1706 are functionalized through the attachment of polypeptides 1708 as shown by the zig-zag line (in place of the antibodies as shown in FIG. 14), such that this structure produces a type of sandwich assay with a ligand 1710, which is shown as the open diamond in the middle. The polypeptides allow precise control of the spring constants to tune the frequency response of the nanomachine. For instance, proline rich structures, alpha helices, or sheets could be attached to the signal influencing element to tune the rigidity of the spring. FIG. 17 shows a general diagram of such a nanomachine.

It is also within the scope of this invention to carry out multiplex detection. Multiplex detection of different target sequences in the same sample can be achieved by using basic signaling nanostructures which produce a different wavelength of fluorescent emission (blue, green, orange, red, etc.). In this case, the detection system would not only be designed to pick up oscillation in target bound signaling nanostructures, but would also observe the sample at different emission wavelengths; i.e. a first target sequence might appear as green blinking signals and the second target sequence might appear as red blinking signals. Heterogeneous multiplexing may also be carried out to take advantage of the spatially independent nature of the nanomachines. By splitting a linear fluidic feed into parallel channels, each with a uniformly distributed lawn of nanostructures (with uniformly distributed colors), multiplexing can be carried across many targets even with the same basic color scheme (by having different lawns in each parallel channel).

### EXAMPLES

### Example 1

### Production of an Oscillatory Nanoscale Signal

The ability to produce an oscillatory signal at the nanoscale has two basic components; turning the system off, and turning the system on. For fluorescence resonant energy transfer (FRET) pairs, this could be the transition from red to green emission, with the green emission being “on” as a result of the free energy introduction and the red emission being in the relaxed “off” state. In another embodiment, a fluorescent nanoparticle/quencher system in the “off” state would be dim due to the quenching activity when in close proximity, and bright when in the “on” state. Preferred embodiments of this invention would maximize the signal change between states. Preferred embodiments would also establish a differences in frequency spectrum (with respect to the kinetic relaxation of the system) between specific and nonspecifically bound molecules.

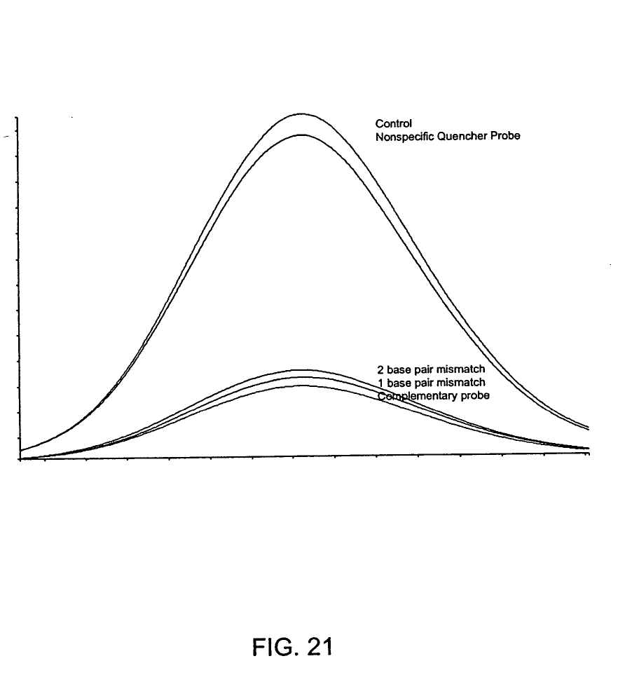

As shown in FIG. 21, the addition of a complementary, one base mismatch, and two base mismatch quencher probes to quantum dots indicate the ability to place the quantum dots in the “off” state. Streptavidin derivitized quantum dots with a 51 base pair capture strand of DNA were hybridized to 20 bp probes modified with QSY-7 quencher in 100 mM sodium phosphate buffer at pH 7.0. Note that all three types of probes bind to the capture sequence on the quantum dot; demonstrating the intrinsic lack of specificity of molecular probes.

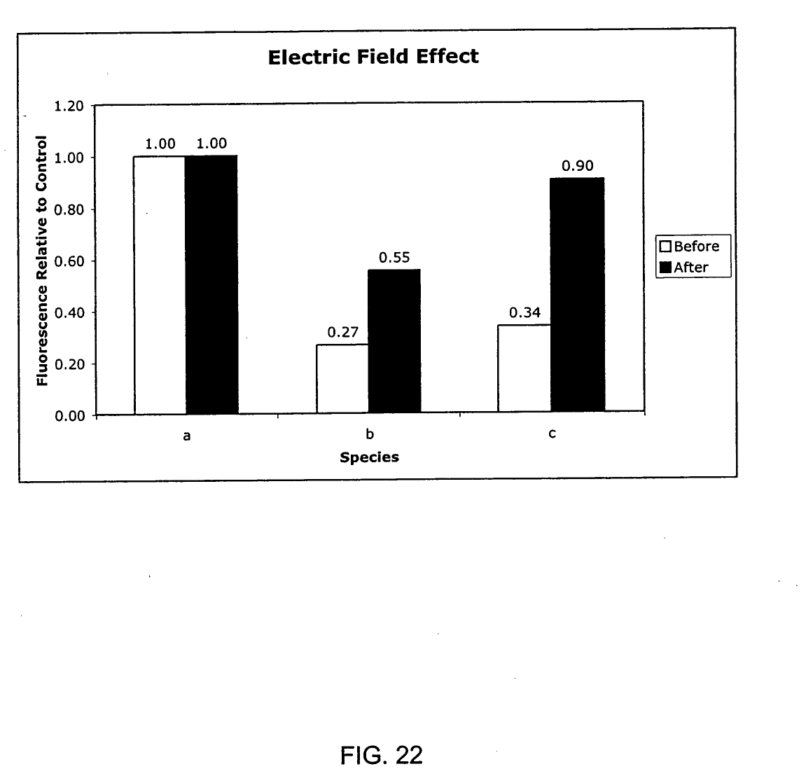

The following is shown in FIG. 22 (Before; white box): [a]Control; Normalized UV transillumination level of streptavidin derivatized quantum dots with capture DNA bound to polymeric biotin embedded in sepharose beads which have been spin coated onto a cellulose acetate membrane (bright). [b,c] Quenched quantum dot system identical to [a], yet with the addition of a 2 bp mismatched 20mer QSY-7 probe (dark, dark). (After; black box) [a.] Normalized Control (bright). [b] Stringency Control; quenched system placed in low salt buffer for ≈5 minutes (dark). [c]. Experimental System; upon placing the membrane containing the nanoconstructs into a transverse electric field for ≈1 minute, it is clear that the quencher has been significantly removed as compared to the control membranes (dark).

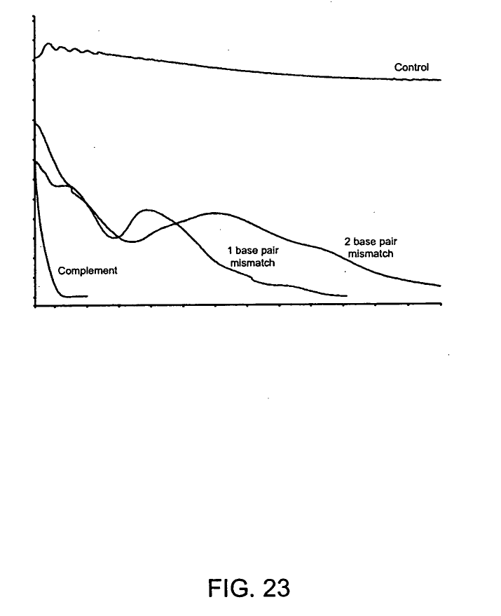

As shown in FIG. 23, once it has been established that many kinds of probes bind to the nanoparticle system, and that it is possible to couple energy into the system to affect the quencher interactions, the question of differential frequency response is of interest. The temporal response of the quanutm dot/QSY-7 system for the three different types of probes is shown in FIG. 23. At time t=0, a bolus of quencher probe is introduced into a cuvette under constant excitation 1=350 nm which contain the quantum dot nanoconstructs. While these curves represent populations of nanoconstructs, they are indicative of the binding kinetics of the individual constituent molecules. These data demonstrate that repeated introduction of free energy into the system will result in very different frequency spectra. It also demonstrates that driving the system at different input frequencies should be able to establish different response bandwidths limited by the relaxation kinetics.

### Example 2

### Nanoparticle Detection of Nucleic Acids in Complex Samples

The identification of unamplified nucleic acid targets has been heretofore nearly impossible using existing assay methods. While it is possible to detect very low levels of fluorescence (single molecule fluorophores or individual fluorescent nano/microparticles) there are few if any assay techniques with the requisite speed, specificity, selectivity and sensitivity which allow low copy number DNA/RNA targets to be detected without prior amplification of the target DNA. Furthermore, most fluorescent systems (molecular beacon probes, FRET probes, etc.) are used to detect PCR amplified DNA targets. Thus, there exists a need for methods of detecting limited quantities of a target nucleic acid without the use of PCR prior to detection.

In one embodiment, methods for identifying a target nucleic acid molecule in a sample are provided. The methods include contacting the target nucleic acid molecule with a first nucleic acid probe comprising a signaling element and contacting the target nucleic acid with a second nucleic acid probe comprising a signal inhibiting element. The second probe hybridizes to the target nucleic acid molecule such that the signal inhibiting element is in proximity to the signaling element thereby reducing the signal associated with the signaling element. Subsequent to hybridization, a pulsed electric field is applied to the nucleic acid complex formed by the target nucleic acid and hybridized probes. The pulsed electric field periodically interrupts the ability of the signal inhibiting element to reduce the signal associated with the signaling element thereby producing an oscillating signal. Such an oscillating signal is easily detectable by numerous methods known to those skilled in the art.

The discussion below refers to the following figures:

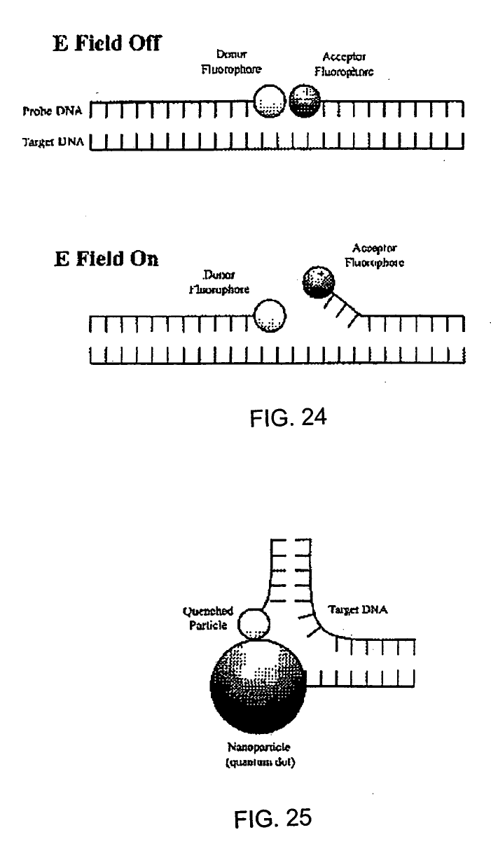

FIG. 24 depicts conventional assay system for fluorescent oscillation via electric fields. Cycling between electric field states should interrupt FRET between a donor and acceptor attached to DNA probes. This combination of spatial detection and deterministic behavior greatly enhances the specificity of the assay.

FIG. 25 depicts an example of a larger fluorescent nanoparticle-target DNA-quencher probe complex (i.e. a “nanomachine”) which produces the oscillating fluorescent signal.

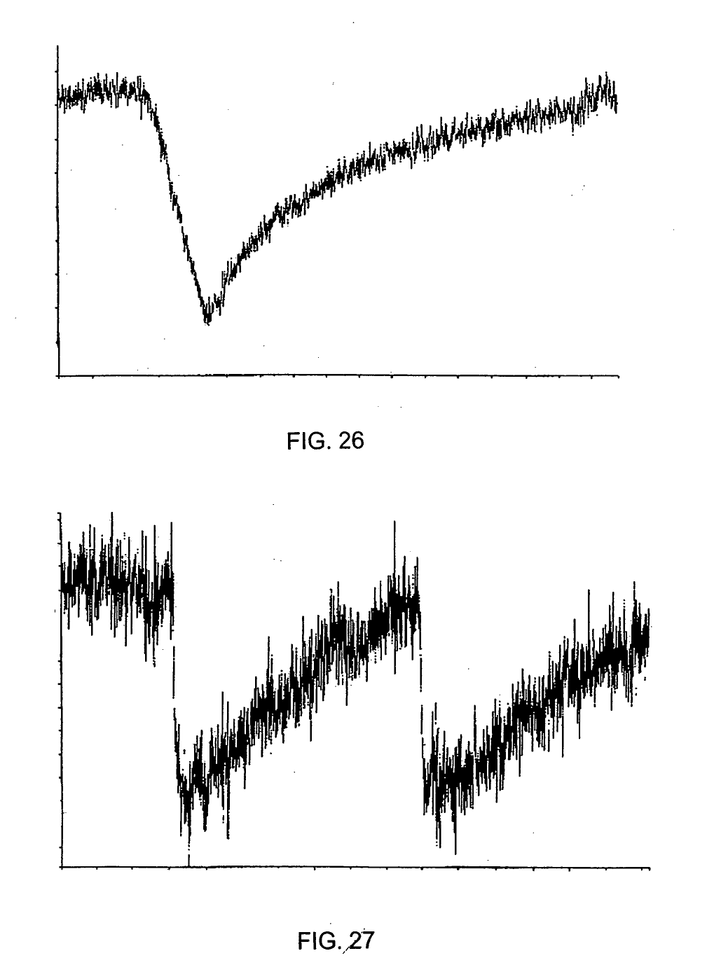

FIG. 26 is a graph depicting an exemplary fluorescence oscillation effect. At about 140 seconds, an electric field is activated which removes the positively charged ethidium bromide from the DNA. At about 210 seconds, the electric field is deactivated and a first order step response recovery is observed. Each leg of the dynamic behavior is well characterized by exponential curves, which indicate linear system behavior.

FIG. 27 is a graph depicting the above the oscillatory behavior of the system given a periodic electric field input. The electric field strength is much higher in FIG. 27 than in FIG. 26, which accounts for the faster drop-off.

As shown in FIG. 24, the signaling element can be a fluorescent label that includes a donor group for fluorescent energy transfer (FRET). The signal inhibiting element can be a fluorescent quencher that includes an acceptor group for fluorescent energy transfer (FRET). In another embodiment, a signal altering second probe may be used instead of a signal inhibiting element, thereby changing the signal produced rather than inhibiting it. In such an embodiment, the signaling element may be a fluorescent nanoparticle (as shown in FIG. 25) that couples to an acceptor group for fluorescent energy transfer (FRET) which acts as the signal altering element.

In one aspect, the application of the electric field results in a change in distance between the signaling element and the signal inhibiting element. The target nucleic acid molecule or nucleic acid probes can be DNA or RNA.

In one aspect, the target nucleic acid molecule can be associated with a pathological condition (such as cancer), an infectious organism or a genetic alteration. The pulsed electric field can be alternating current or direct current. The signaling element can be a nanoparticle, such as a polymer bead, a quantum dot or a gold particle.

In another aspect, the sample is associated with a solid support. The solid support can be an array, such as a microarray.

In another embodiment, a diagnostic profile produced by a method of the invention is provided. Such a profile can be correlated with a wild-type state, a pathological condition, or a genetic alteration is a subject from which a sample is obtained.

Accordingly, one embodiment of the invention comprises a novel electric field mechanism by which a combination of a fluorescent nanoparticle (i.e., polymer bead, quantum dot, gold particle) and quencher (or fluorescent) probe can be used to rapidly detect very low levels of target DNA/RNA sequences in complex samples (homogeneous or heterogeneous formats, microarray). One example of the technique involves the use of fluorescent nanoparticles and quencher DNA probes that selectively hybridize to a specific target DNA sequence. Once hybridized, any fluorescent nanoparticle-target DNA-quencher probe combinations will have a reduced fluorescence signal. Upon applying a pulsed electric field (DC or AC) to the sample, the fluorescent nanoparticle-target DNA-quencher probe complex will be altered and produce an oscillating fluorescent signal as a direct result of the applied field. These oscillating fluorescent nanoparticle complexes can now be spatially resolved and easily detected among the thousands of non-hybridized or partially hybridized fluorescent nanoparticles using a fluorescence imaging system and temporal signal processing techniques. In homogenous hybridization assay formats, the fact that a large number of fluorescent nanoparticles and quencher probes can be used means that the hybridization kinetics will also be greatly accelerated. Thus, this novel mechanism provides speed, high sensitivity and specificity for carrying out DNA hybridization assays without the need to use prior amplification of the target DNA.

In another embodiment, a diagnostic profile produced by a method of the invention is provided. Such a profile can be correlated with a genetic wild type, mutant or heterozygous state, or other polymorphic genetic marker, gene expression level or presence of an infectious agent, a protein, ligand, antibody, antigen, or biomarker. In another aspect, the sample is associated with a cellular support. The cellular support can be an in situ hybridization. In another aspect, the sample is associated with a solid support. The solid support can be an array, such as a microarray.

The present invention provides a fluorescent technique that allows truly low level targets to be rapidly detected without prior amplification. Specifically, the invention provides an opportunity to identify limited amounts of target nucleic acid molecules by spatially resolving them from non-target sequences in an assay. Essentially, the targeted molecules are identified because the application of a pulsed electric field causes the fluorescent particles associated with the hybridized probes to “blink” (i.e., oscillate from a fluorescent to non-fluorescent state). Rather than quantifying bulk changes in fluorescence, the oscillating quenching and emission of the fluorescent particle-target nucleic acid molecule-quencher probe complexes can be identified even in fields with very large number of other fluorescent particles. By way of an example from astronomy, the present analysis is somewhat akin to how “pulsars or neutron stars”, which produce fluctuating light intensities (blinking) are resolved among huge numbers of other stars.

As previously noted, almost all present homogenous (in solution) and heterogeneous (solid supports, dot blots, DNA microarrays, biochips, etc.) DNA hybridization assays require prior amplification of the target DNA. Present uses of most fluorophores, FRET systems, molecular beacons, fluorescent nanoparticles, gold particles and new fluorescent quantum dots are used to detect amplified DNA sequences.

The inventors have demonstrated that single-stranded DNA and a positively charged fluorophore (ethidium bromide) exhibit reproducible first order, linear system behavior when under the direct influence of high DC electric fields (see FIGS. 26 and 27). The extension to other linear systems is straightforward, i.e. given a periodic signal of appropriate frequency such that it doesn't band limit the system, we can predict the outcome of the experiment with linear systems theory.

The invention encompasses the use of nanoparticles. Nanoparticles include nanoshells as disclosed in U.S. Pat. No. 6,344,272 (incorporated by reference), metal colloids as disclosed in U.S. Pat. No. 5,620,584 272 (incorporated by reference), fullerenes and derivatized fullerenes, as disclosed in U.S. Pat. Nos. 5,739,376; 6,162,926; 5,994,410, all of which are incorporated by reference, as well as nanotubes including single walled nanotubes, as disclosed in U.S. Pat. No. 6,183,714 (incorporated by reference), which can also be derivatized.

In one example, DNA quencher probes and fluorophore probes, target DNA sequences, and primers are obtained in order to identify mutations in p53 axon 8 gene. Subsequent to hybridization, an electric filed is used to produce fluorescent oscillations in the hybridized complexes. The “pulse” signal is indicative of hybridization.

The present invention also provides fluorescent nanoparticle (quantum dot) based systems. Additional embodiments of the invention include fluorescent resonant energy transfer (FRET) complexes, time resolved lanthanide complexes, use of DC and AC fields to rotate or spin gold or other particles for reflecting light, use magnetic type nanoparticles and use of other bioaffinity agents such a proteins, antibodies, etc. The invention also has applications in the creation of nanophotonic mechanisms and devices which have a wide variety of computational or data storage applications.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A method comprising binding a nanostructure and an associated structure to a target and reversibly altering interaction between the nanostructure, the associated structure and the target.

2. The method of claim 1, wherein the reversible alteration is in response to applied energy.

3. The method of claim 2, wherein the applied energy is an electric field.

4. The method of claim 2, wherein the applied energy is a DC field.

5. The method of claim 2, wherein the applied energy is an AC field.

6. The method of claim 2, wherein the applied energy is a capacitive field.

7. The method of claim 2, wherein the applied energy is thermal.

8. The method of claim 2, wherein the applied energy is electrical.

9. The method of claim 2, wherein the applied energy is chemical.

10. The method of claim 9, wherein the chemical energy is adenosine triphosphate (ATP) or nicotinamide adenine dinucleotide (NADH).

11. The method of claim 2, wherein the applied energy is photonic.

12. The method of claim 2, wherein the applied energy is magnetic.

13. The method of claim 2, wherein the applied energy is kinetic.

14. The method of claim 2, wherein the applied energy is acoustic.

15. The method of claim 14, wherein the applied energy is ultrasonic.

16. The method of claim 2, wherein the applied energy is microwave.

17. The method of claim 2, wherein the applied energy is radiative.

18. The method of claim 1, wherein the reversible alteration is deformation.

19. The method of claim 18, wherein the deformation is elastic, inelastic or plastic deformation.

20. The method of claim 1, wherein the reversible alteration is angular motion.

21. The method of claim 1, wherein the reversible alteration is a separation distance.

22. The method of claim 1, wherein the reversible alteration is a rotation.

23. The method of claim 1, wherein the reversible alteration is a linear displacement.

24. The method of claim 1, wherein the reversible alteration is helical motion.

25. The method of claim 1, wherein the reversible alteration is in response to shear force.

26. The method of claim 1, wherein the reversible alteration is in response to pressure.

27. The method of claim 1, wherein the interaction is resonant energy.

28. The method of claim 27, wherein the resonant energy is dipole coupling.

29. The method of claim 27, wherein the resonant energy is quadrapole coupling.

30. The method of claim 27, wherein the resonant energy is fluorescence resonance energy transfer.

31. The method of claim 1, wherein the interaction is plasmonic.

32. The method of claim 1, wherein the interaction is near field coupling.

33. The method of claim 1, wherein the interaction is photonic.

34. The method of claim 1, wherein the interaction is capacitive.

35. The method of claim 1, wherein the interaction is magnetic.

36. The method of claim 1, wherein the interaction is electrostatic.

37. The method of claim 1, wherein the method further comprises detecting a changed characteristic resulting from the interaction.

38. The method of claim 37, wherein the changed characteristic is a variation in luminescence.

39. The method of claim 37, wherein the changed characteristic is a variation in fluorescence.

40. The method of claim 37, wherein the changed characteristic is a variation in optical properties.

41. The method of claim 37, wherein the changed characteristic is color.

42. The method of claim 37, wherein the changed characteristic is a magnetic field.

43. The method of claim 37, wherein the changed characteristic is an electric field.

44. Then method of claim 37, wherein the changed characteristic is a surface enhanced Raman scattering (SERS) or Raman spectra.

45. The method of claim 1, wherein the reversibly altering interaction between the nanostructure, the associated structure and the target is spatially independent.

46. The method of claim 45, wherein the interaction occurs in a solution.

47. The method of claim 45, wherein the interaction occurs at a fixed location for which there is no a priori knowledge.

48. The method of claim 45, wherein the interaction occurs in a homogeneous assay.

49. The method of claim 45, wherein the interaction occurs in a heterogeneous assay.

50. The method of claim 45, wherein the interaction occurs in an in situ assay.

51. The method of claim 45, wherein the interaction occurs at a fixed location for which there is a priori knowledge.

52. The method of claim 51, wherein the method is performed on a microarray or nanoarray.

53. An apparatus comprising a nanostructure and an associated structure, wherein the nanostructure and the associated structure are adapted to reversibly interact with each other and a target.

54. The apparatus of claim 53, wherein the nanostructure is a quantum dot.

55. The apparatus of claim 53, wherein the nanostructure is a semiconductor nanoparticle.

56. The apparatus of claim 53, wherein the nanostructure is a photonic crystal.

57. The apparatus of claim 53, wherein the nanostructure is a metallic nanoparticle.

58. The apparatus of claim 53, wherein the nanostructure is a ceramic nanoparticle.

59. The apparatus of claim 53, wherein the nanostructure is a polymeric nanoparticle.

60. The apparatus of claim 53, wherein the nanostructure is a nanotube.

61. The apparatus of claim 53, wherein the associated structure is a quantum dot.

62. The apparatus of claim 53, wherein the associated structure is a semiconductor nanoparticle.

63. The apparatus of claim 53, wherein the associated structure is a photonic crystal.

64. The apparatus of claim 53, wherein the associated structure is a metallic nanoparticle.

65. The apparatus of claim 53, wherein the associated structure is a ceramic nanoparticle.

66. The apparatus of claim 53, wherein the associated structure is a polymeric nanoparticle.

67. The apparatus of claim 53, wherein the associated structure is a nanotube.

68. The apparatus of claim 53, wherein the associated structure further comprises a fluorophore, a quencher, a chromophore, a phycobillic protein, a lumiphore, a fluorescent protein.

69. The apparatus of claim 53, wherein the apparatus further comprises an interaction amplifying element.

70. The apparatus of claim 69, wherein the interaction amplifying element is attached to the nanostructure.

71. The apparatus of claim 69, wherein the interaction amplifying element is attached to the associated structure.

72. The apparatus of claim 69, wherein the interaction amplifying element is a pressure responsive element.

73. The apparatus of claim 69, wherein the interaction amplifying element is a displacement amplifying element.

74. The apparatus of claim 53, wherein the nanostructure has attached thereto a fluorescent donor, and wherein the associated structure further comprises a fluorescent quencher.

75. The apparatus of claim 53, wherein the nanostructure and the associated structure have attached thereto individual members of a fluorescent energy transfer (FRET) pair.

76. The apparatus of claim 53, wherein the nanostructure and the associated structure are adapted to reversibly and spatially independently interact with each other and a target.

77. The apparatus of claim 76, wherein the nanostructure and the associated structure are adapted to reversibly and spatially independently interact with each other and a target in solution.

78. The apparatus of claim 76, wherein the nanostructure and the associated structure are adapted to reversibly and spatially independently interact with each other and a target on a surface.

79. The apparatus of claim 78, wherein the surface is on a microarray or nanoarray.

80. A method comprising:
a. providing nanostructures, associated structures, and targets; and
b. detecting a temporally varying, spatially independent, information signal produced by the nanostructures, associated structures, and targets.

81. The method of claim 80, further comprising applying a driving force to the nanostructures, associated structures, and targets.

82. The method of claim 81, wherein the driving force is photonic.

83. The method of claim 81, wherein the driving force is electrical.

84. The method of claim 81, wherein the driving force is thermal.

85. The method of claim 81, wherein the driving force is magnetic.

86. The method of claim 81, wherein the driving force is periodic.

87. The method of claim 81, wherein the driving force is a series of impulses.

88. The method of claim 81, wherein the driving force is an impulse.

89. The method of claim 81, wherein the driving force is constant.

90. The method of claim 80, wherein the information signal is a variation in fluorescence of the nanostructure, associated structure, and target combinations.

91. The method of claim 80, wherein the information signal is a variation in color of the nanostructure, associated structure, and target combinations.

92. The method of claim 80, wherein the information signal is a variation in temperature of the nanostructure, associated structure, and target combinations.

93. The method of claim 80, wherein the information signal is a variation in electric field strength of the nanostructure, associated structure, and target combinations.

94. The method of claim 80, wherein the information signal is a variation in magnetic field strength of the nanostructure, associated structure, and target combinations.

95. The method of claim 80, wherein the information signal is a change in frequency of a characteristic of the nanostructure, associated structure, and target combinations.

96. The method of claim 80, further comprising processing the detected information signal to classify a molecular binding event.

97. The method of claim 80, further comprising processing the detected information signal utilizing neural networks.

98. The method of claim 80, further comprising processing the detected information signal utilizing Bayesian networks.

99. The method of claim 80, further comprising processing the detected information signal utilizing MAP detection.

100. The method of claim 80, further comprising applying a driving force that produces a reversibly altering interaction between a nanostructure, an associated structure, and a target comprising the information signal.

101. A system for detecting a target comprising:
a. a nanostructure, an associated structure, and the target, adapted to produce a reversibly altering interaction between the nanostructure, associated structure and target;
b. an input source adapted to impart energy to the nanostructure, associated structure, and target combination thereby producing the reversibly altering interaction; and
c. a detector configured to detect transduced output generated by the reversibly altering interaction.

102. The system of claim 101, wherein the imparted energy is photonic.

103. The system of claim 101, wherein the imparted energy is electrical.

104. The system of claim 101, wherein the imparted energy is thermal.

105. The system of claim 101, wherein the imparted energy is magnetic.

106. The system of claim 101, wherein the imparted energy is periodic.

107. The system of claim 101, wherein the imparted energy is a series of impulses.

108. The system of claim 101, wherein the imparted energy is an impulse.

109. The system of claim 101, wherein the imparted energy is constant.

110. The system of claim 101, wherein the transduced output is a variation in fluorescence of the nanostructure, associated structure, and target.

111. The system of claim 101, wherein the transduced output is a variation in color of the nanostructure, associated structure, and target.

112. The system of claim 101, wherein the transduced output is a variation in temperature of the nanostructure, associated structure, and target.

113. The system of claim 101, wherein the transduced output is a variation in a frequency of a characteristic of the nanostructure, associated structure, and target.

114. The system of claim 101, wherein the transduced output is a variation in electrical field strength of the nanostructure, associated structure, and target.

115. The system of claim 101, wherein the transduced output is a variation in magnetic field strength of the nanostructure, associated structure, and target.

116. A signaling nanostructure comprising at least one target binding region and at least one signal influencing region, wherein the signal influencing region has attached thereto a signal influencing element that alters a signaling characteristic of the nanostructure, and wherein the target binding region is selective for a predetermined target.

117. The signaling nanostructure of claim 116, wherein the signal influencing element is a signal inhibiting element.

118. The signaling nanostructure of claim 116, wherein the signaling nanostructure is fluorescent and the signal inhibiting element is a fluorescent quencher.

119. The signaling nanostructure of claim 116, wherein the target binding region and the signal influencing region are asymmetrically patterned on the surface of the signaling nanostructure.

120. The signaling nanostructure of claim 116, wherein the nanostructure further comprises only one target binding region.

121. The signaling nanostructure of claim 116, wherein the signal influencing element is a metallic nanoparticle.

122. A signaling nanostructure having at least one signal influencing region having attached thereto a signal influencing element that alters a signaling characteristic of the nanostructure, wherein the signal influencing element further comprises at least one target binding region having attached thereto a target binding element, and wherein the target binding element is selective for a predetermined target.

123. The signaling nanostructure of claim 121, wherein the signal influencing element is a metallic nanoparticle.

124. The signaling nanostructure of claim 122, wherein the target binding region further comprises a single target binding element attached thereto.

125. The signaling nanostructure of claim 122, wherein the target binding element is an oligonucleotide.

126. The signaling nanostructure of claim 122, wherein the target binding element is an antibody.

127. The signaling nanostructure of claim 122, wherein the target binding element is a polypeptide.

128. The signaling nanostructure of claim 122, wherein the signaling nanostructure and the signal influencing element are attached via the target binding element.

129. The signaling nanostructure of claim 122 having a first signal influencing element attached thereto, wherein the signal influencing element further comprises at least one target binding region, and wherein the signaling nanostructure further comprises a second signal influencing element attached thereto.

130. The signaling nanostructure of claim 122, wherein the first and the second signal influencing elements are metallic nanoparticles.

131. A kit comprising:
a. a first signaling nanostructure having a first metallic nanoparticle attached thereto, wherein the first metallic nanoparticle further comprises at least one first target binding region having attached thereto a first target binding element, and wherein the first target binding element is selective for a predetermined target; and
b. a second metallic nanoparticle comprising at least one second target binding region having attached thereto a second target binding element, and wherein the second target binding element is selective for the same predetermined target.

132. The kit of claim 131, wherein the first and second target binding elements are antibodies.

133. The kit of claim 131, wherein the first and second target binding elements are oligonucleotides.

134. The kit of claim 131, wherein the first and second target binding elements are polypeptides.

135. The kit of claim 131, wherein the first and second metallic nanoparticle are attached via a tethering group.

136. The kit of claim 131, wherein the tethering group is a synthetic polymer, a single stranded nucleic acid, a fatty acid, a glycosaminoglycan or a polypeptide.

137. A method comprising the steps of:
a. binding a nanostructure to a target;
b. binding an associated structure to the target;
c. reversibly altering an interaction between the nanostructure, the associated structure and the target to produce information; and
d. detecting the information.

138. The method of claim 137, wherein the target is a nucleic acid.

139. The method of claim 137, wherein the target is a protein.

140. The method of claim 137, wherein the target is an inorganic surface.

141. The method of claim 137, wherein the target is genomic nucleic acid.

142. The method of claim 137 further comprising detecting a target in a biological sample.

143. The method of claim 137, wherein the biological sample is a cell or tissue sample on a microscope slide.

144. The method of claim 143, wherein the target is a nucleic acid.

145. The method of claim 137, wherein the nanostructure further comprises a first target binding region having a target binding element attached thereto that is selective for a predetermined target; and wherein the associated structure further comprises a second target binding region having attached thereto a second target binding element that is selective for the same predetermined target.

146. The method of claim 145, wherein the first and second target binding elements are oligonucleotides.

147. The method of claim 137, wherein the target is an antigen, and wherein the first and second target binding elements are antibodies that bind to the antigen.

148. The method of claim 137 adapted to be performed in a solution, and wherein step c. is performed without removing the nanostructure or the associated structure from the solution.

149. A metallic nanoparaticle having attached thereto a signaling element and a target binding element.

150. The metallic nanoparticle of claim 149, wherein the signaling element is a quantum dot.

151. The metallic nanoparticle of claim 149, wherein the signaling element is a fluorophore.

152. The metallic nanoparticle of claim 149, wherein the signaling element is a FRET donor or a FRET acceptor.

153. The metallic nanoparticle of claim 149, wherein the target binding element is an antibody.

154. The metallic nanoparticle of claim 149, wherein the target binding element is a nucleic acid.

155. The metallic nanoparticle of claim 149, wherein the target binding element is a polypeptide.

156. A method for identifying a target nucleic acid molecule in a sample, the method comprising:
a. contacting the target nucleic acid molecule with a first nucleic acid probe comprising a signaling element, wherein the first nucleic acid probe hybridizes to the target molecule;
b. contacting the target nucleic acid with a second nucleic acid probe comprising a signal inhibiting element, wherein the second probe hybridizes to the target nucleic acid molecule such that the signal inhibiting element is in proximity to the signaling element thereby reducing the signal associated with the signaling element;
c. applying a pulsed electric field to a nucleic acid complex formed by the target nucleic acid and hybridized probes, wherein the pulsed electric field periodically interrupts the ability of the signal inhibiting element to reduce the signal associated with the signaling element thereby producing an oscillating signal; and
d. detecting the oscillating signal.

157. The method of claim 156, wherein the signaling element is a fluorescent label.

158. The method of claim 156, wherein the signal inhibiting element is a fluorescent quencher.

159. The method of claim 157, wherein the fluorescent label comprises a donor group for fluorescent energy transfer (FRET).

160. The method of claim 158, wherein the fluorescent quencher comprises an acceptor group for fluorescent energy transfer (FRET).

161. The method of claim 156, wherein the application of the electric field results in a change in distance between the signaling element and the signal inhibiting element.

162. The method of claim 156, wherein the target nucleic acid molecule is DNA or RNA.

163. The method of claim 156, wherein the first nucleic acid probe is DNA or RNA.

164. The method of claim 156, wherein the second nucleic acid probe is DNA or RNA.

165. The method of claim 156, wherein the target nucleic acid molecule is associated with a pathological condition or genetic alteration.

166. The method of claim 156, wherein the sample comprises a plurality of non-target nucleic acid molecules.

167. The method of claim 156, wherein the sample comprises a plurality of target nucleic acid molecules.

168. The method of claim 156, wherein the pulsed electric field is alternating current or direct current.

169. The method of claim 156, wherein the signaling element is a nanoparticle.

170. The method of claim 169, wherein the nanoparticle is selected from the group consisting of a polymer bead, a quantum dot and a gold particle.

171. The method of claim 156, wherein the sample is associated with a solid support.

172. The method of claim 171, wherein the solid support is an array.

173. The method of claim 172, wherein the array is a microarray.

174. The method of claim 156, further comprising amplifying the target nucleic acid molecule.

175. A diagnostic profile produced by the method of claim 156.

176. The diagnostic profile of claim 175, wherein the diagnostic profile is correlated with a wild-type state, a pathological condition, or a genetic alteration.
